(19) United States
(12) Patent Application Publication
(10) Pub. No.: US 2007/0082898 A1
(43) Pub. Date: Apr. 12, 2007

(54) 5 Amino-2-carbonylthiophene derivatives for use as p38 map kinase inhibitors in the treatment of inflammatory diseases

(75) Inventors: Adrian Liam Gill, Cambridge (GB); Steven Woodhead, Cambridge (GB); Maria Carr, Cambridge (GB)

Correspondence Address: HESLIN ROTHENBERG FARLEY & MESITI PC, 5 COLUMBIA CIRCLE, ALBANY, NY, 12203, US

(73) Assignee: Astex Therapeutics Limited, Cambridge (GB)

(21) Appl. No.: 10/553,361

(22) PCT Filed: Apr. 13, 2004
(86) PCT No.: PCT/GB04/01589

§ 371 Date: Oct. 31, 2006

(30) Foreign Application Priority Data
Apr. 14, 2003 (GB) 0308511.5

(51) Int. Cl. A61K 31/538; A61K 31/522; A61K 31/52; A61K 31/517; A61K 31/496; A61K 31/4745; A61K 31/4709; A61K 31/4178; A61K 31/416; C07D 417/02; C07D 413/02; C07D 409/02

(52) U.S. Cl. 514/230.5; 514/248; 514/249; 514/263.23; 514/266.2; 514/300; 514/241; 514/256; 514/307; 514/314; 514/362; 514/374; 514/365; 514/397; 514/381; 514/406; 514/372; 514/364; 514/447; 544/105; 544/276; 544/277; 544/236; 544/238; 544/314; 544/331; 544/353; 544/284; 546/122; 546/148; 546/159; 549/68; 548/190; 544/209; 548/215; 548/311.1

(57) ABSTRACT

The invention provides the use of a compound for the manufacture of a medicament for the prophylaxis or treatment of a disease state or condition mediated by a p38 MAP kinase; the compound being defined by formula (I): wherein: R¹ and R² are the same or different and each is selected from hydrogen, C₁₋₄ hydrocarbyl, halogen and cyano; X is selected from C═O, C═S, C(═O)NH, C(═S)NH, C(═O)O, C(═O)S, C(═S)O and C(═S)S; R³ is selected from aryl and hetcroaryl groups each having from 5 to 12 ring members, the aryl and heteroaryl groups each being unsubstituted or substituted by one or more substituent groups R⁷ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, 0, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; X¹ is O, S or NR^{c} and X² is ═O, ═S or ═NR^{c}; R^{c} is hydrogen or C₁₋₄ hydrocarbyl; R⁴ is a group YR⁵ or a group R⁶; Y is is NH, O or S; R⁵ is selected from (a) carbocyclic and heterocyclic groups having from 3 to 12 ring members; and (b) C₁₋₈ hydrocarbyl groups optionally substituted by one or more substituents selected from hydroxy, oxo, halogen, cyano, amino, mono- or di- C₁₋₄ hydrocarbylamino, and carbocyclic and heterocyclic groups having from 3 to 12 ring members, wherein one or more carbon atoms of the C₁₋₈ hydrocarbyl group may optionally be replaced by O, S, SO, SO₂, NR^{c}, X¹C(X²), C(X²)X¹ or X¹C(X²)X¹, provided that when Y is O, a carbon atom adjacent to the group Y is not replaced by O; and R⁶ is a heterocyclic group having from 4 to 12 ring members and containing at least one ring nitrogen atom through which R⁶ is linked to the adjacent carbonyl group; wherein the carbocyclic and heterocyclic groups of substituents R⁵ and R⁶ are each unsubstituted or substituted by one or more substituent groups R⁷ as hereinbefore defined. Also provided are novel compounds, pharmaceutical compositions containing the compounds and methods for their preparation.

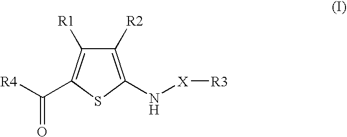

This invention relates to compounds that inhibit or modulate the activity of p38 MAP kinase and to the use of the compounds in the treatment or prophylaxis of disease states or conditions mediated by p38 MAP kinase. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signaling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signaling pathways to activate or inactivate (either directly or indirectly), for example, a metabolic enzyme, regulatory protein, receptor, cytoskeletal protein, ion channel or pump, or transcription factor. Disruption of intracellular signal transduction due to defective control of protein phosphorylation has been implicated in a number of diseases, including, for example, inflammation, cancer, allergy/asthma, disease and conditions of the immune system, disease and conditions of the central nervous system, and angiogenesis.

The mitogen-activated protein (MAP) kinase family consists of a series of structurally related proline-directed serine/threonine kinases that are activated either by growth factors (such as EGF) and phorbol esters (ERK), or by IL-1, TNF or stress (p38, JNK). These kinases mediate the effects of numerous extracellular stimuli on a wide array of biological processes, such as cell proliferation, differentiation and death. Three groups of mammalian MAP kinases have been studied in detail: the extracellular signal-regulated kinases (ERK), the c-Jun NH₂— terminal kinases (JNK) and the p38 MAP kinases.

There are five known human isoforms of p38 MAP kinase, p38α, p38β, p38β2, p38γ and p38δ. The p38 kinases, which are also known as cytokine suppressive anti-inflammatory drug binding proteins (CSBP), stress activated protein kinases (SAPK) and RK, are responsible for phosphorylating (Stein et al., Ann. Rep. Med Chem., 31, 289-298 (1996)) and activating transcription factors (such as ATF-2, MAX, CHOP and C/ERPb) as well as other kinases (such as MAPKAP-K2/3 or MK2/3), and are themselves activated by physical and chemical stress (e.g. U.V, osmotic stress), pro-inflammatory cytokines and bacterial lipopolysaccharide (LPS) (Herlaar, E & Brown, Z., Molecular Medicine Today, 5: 439-447 (1999)). The products of p38 phosphorylation have been shown to mediate the production of inflammatory cytokines, including TNF and IL-1, and cyclooxygenase-2 (COX-2). Each of these cytokines has been implicated in numerous disease states and conditions. IL-1 and TNF are also known to stimulate the production of other proinflammatory cytokines such as IL-6 and IL-8.

Interleukin-1 (IL-1) and Tumor Necrosis Factor (TNF) are biological substances produced by a variety of cells, such as monocytes or macrophages. IL-1 has been demonstrated to mediate a variety of biological activities thought to be important in immunoregulation and other physiological conditions such as inflammation (e.g. Dinarello, et al., Rev. Infect. Disease, 6: 51 (1984)). The myriad of known biological activities of IL-1 include the activation of T helper cells, induction of fever, stimulation of prostaglandin or collagenase production, neutrophil chemotaxis, induction of acute phase proteins and the suppression of plasma iron levels.

There are many disease states in which excessive or unregulated IL-1 production is implicated in exacerbating and/or causing the disease. These include rheumatoid arthritis (Arend et al., Arthritis & Rheumatism 38(2): 151-160, osteoarthritis, endotoxemia and/or toxic shock syndrome, other acute or chronic inflammatory disease states such as the inflammatory reaction induced by endotoxin or inflammatory bowel disease; tuberculosis, atherosclerosis, Hodgkin's disease (Benharroch et al., Euro. Cytokine Network 7(1): 51-57), muscle degeneration, cachexia, psoriatic arthritis, Reiter's syndrome, gout, traumatic arthritis, rubella arthritis, acute synovitis and Alzheimer's disease. Evidence also links IL-1 activity to diabetes and pancreatic B cells (Dinarello, J. Clinical Immunology, 5: 287-297 (1985)). Because inhibition of p38 leads to inhibition of IL-1 production, it is envisaged that p38 inhibitors will be useful in the treatment of the above listed diseases.

Excessive or unregulated TNF production has been implicated in mediating or exacerbating a number of diseases including rheumatoid arthritis (Maini et al., APMIS, 105(4): 257-263), rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, fever and myalgias due to infection, such as influenza, herpes simplex virus type-1 (HSV-1), HSV-2, cytomegalovirus (CMV), varicella-zoster virus (VZV), Epstein-Barr virus (EBV), human herpes virus-6 (HHV-6), HHV-7, HHV-8, pseudorabies, rhinotracheitis and cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, or pyresis. Because inhibition of p38 leads to inhibition of TNF production, it is envisaged that p38 inhibitors will be useful in the treatment of the above listed diseases.

Interleukin-8 (IL-8) is a chemotactic factor produced by several cell types including mononuclear cells, fibroblasts, endothelial cells, and keratinocytes. Its production from endothelial cells is induced by IL-1, TNF, or lipopolysaccharide (LPS). IL-8 stimulates a number of functions in vitro. It has been shown to have chemoattractant properties for neutrophils, T-lymphocytes, and basophils. In addition it induces histamine release from basophils from both normal and atopic individuals as well as lysozomal enzyme release and respiratory burst from neutrophils. IL-8 has also been shown to increase the surface expression of Mac-1 (CD 11 blCD 18) on neutrophils without de novo protein synthesis; this may contribute to increased adhesion of the neutrophils to vascular endothelial cells. Many diseases are characterized by massive neutrophil infiltration. Conditions associated with an increased in IL-8 production (which is responsible for chemotaxis of neutrophils into the inflammatory site) would benefit from treatment with compounds which are suppressive of IL-8 production. Recently Chronic Obstructive Pulmonary Disease (COPD) has been linked to raised levels of IL-8 and neutrophil infiltration of the lung (Barnes et al., Curr. Opin. Pharmacol., 1: 242-7 (2001)). Other conditions linked to IL-8 include acute respiratory distress syndrome (ARDS), asthma, pulmonary fibrosis and bacterial pneumonia.

IL-1 and TNF affect a wide variety of cells and tissues and these cytokines as well as other leukocyte derived cytokines are important and critical inflammatory mediators of a wide variety of disease states and conditions. The inhibition of these cytokines is of benefit in controlling, reducing and alleviating many of these disease states.

Inhibition of signal transduction via p38, which in addition to IL-1, TNF and IL-8 described above is also required for the synthesis and/or action of several additional pro-inflammatory proteins (i.e., IL-6, GM-CSF, COX-2, collagenase and stromelysin), is expected to be a highly effective mechanism for regulating the excessive and destructive activation of the immune system. This expectation is supported by the potent and diverse anti-inflammatory activities described for p38 kinase inhibitors (Badger, et al., J. Pharm. Exp. Thera., 279:1453-1461(1996); Griswold, et al., Pharmacol. Comm., 7: 323-229 (1996)).

WO 00/71535 (Scios Inc.) discloses indole-type compounds as inhibitors of p38 kinase.

WO 93/14081 (Smith-Kline Beecham) discloses 1,3,4-triaryl imidazoles as inhibitors of p38 MAP kinase.

WO 99/15164 (Zeneca) discloses various bis-benzamidophenyl derivatives compounds which exhibit inhibition of p38 activity.

WO 99/32111 and WO 99/32463 (Bayer) each disclose series of diarylurea compounds which act as p38 MAP kinase inhibitors.

WO 99/00357 (Vertex) discloses a further class of diarylurea compounds as p38 MAP kinase inhibitors.

EP 1253142 discloses various heteroaryl compounds as thrombopoietin receptor agonists.

WO 01/40223 discloses a class of pesticidal substituted aminoheterocyclylamides.

An article by A. R. Redman et al, in Bioorganic & Medicinal Chemistry Letters, 11, 9-12, (2001) describes thienyl compounds, in particular thienyl ureas, having p38 kinase inhibitory activity. The compounds disclosed in Redman et al are characterised by the presence of an aryl ureido group at the 3-position of the thiophene ring.

WO 03/004020 (Boehringer Ingelheim) discloses a class of heteroaryl diamides in which one amide group contains a phenyl, pyridyl or pyrimidinyl group having a carbocyclic or heterocylic group bonded to the ortho position thereof either directly or through an intervening linker atom or group. The compounds are described as being inhibitors of the microsomal triglyceride transfer protein and therefore useful in lowering plasma lipoprotein levels.

WO 96/41795 (Fujisawa) discloses thiophene diamides that are useful as vasopressin antagonists.

WO 94/04525 (Otsuka) discloses benzazepines and aza analogues in which a nitrogen atom of the benzazepine group is attached to an amide group that can contain a heterocyclic ring such as a thiophene. The compounds are vasopressin and oxytocin antagonists.

EP 0 592 167 (Zeneca) describes antibiotic thiopenem derivatives containing an optionally N-substituted pyrrolidine ring that can be linked via an amide bond to a thiophene group.

A. Khalaf et al. Tetrahedron, (2000), 56 (29), 5225-5239 describes a thiophene diamide containing a 5-nitro-2-thiophenyl group. The compound is stated to be a DNA minor groove binder.

JP 10212271 (Zeria) (Chem. Abstract 129:202763) describes a class of compounds that are useful in the treatment of digestive tract disorders. The compounds are amides that can contain a thiophene carboxylic acid amide group. Also disclosed as intermediates are the corresponding carboxylic acid esters.

JP 05230009 (Taisho) discloses as inhibitors of Platelet-Activating Factor (PAF) compounds, N-substituted amides of 5-(4-carbamimidoyl-benzoylamino)-thiophene-2-carboxylic acid. The amide N-substituent groups contain an alkylene chain terminating in a carboxylic acid or alkoxycarbonyl group.

Gewald et al., J. für Prakt. Chem., (Leipzig), (1991), 333(2), 229-36 describes the reactions of 2-aminothiophene-3-carbonitriles with heterocumulenes. The article discloses a urea, each nitrogen atom of which bears a 2-ethoxycarbonyl-3-methyl-4-cyanothien-2yl group.

U.S. Pat. No. 4,767,758 (CNDR) describes thiophene analogues that are useful in treating tumours. The thiophenes can contain amide substituents.

U.S. Pat. No. 5,571,810 (Fujisawa) describes 2,3-diaryl thiophenes that have anti-inflammatory and analgesic activity and which are considered to be useful in treating a range of diseases including rheumatoid arthritis.

U.S. Pat. No. 6,414,013 (Pharmacia & Upjohn) discloses 3-aminocarbonyl-2-carboxamidothiophenes that have activity as kinase inhibitors and which are considered to be useful in the treatment of a variety of diseases including cancers, arthritis and autoimmune diseases.

WO 99/32477 (Schering) discloses ortho-anthranilamide derivatives as anti-coagulants.

### SUMMARY OF THE INVENTION

The present invention provides a further class of compounds that have p38 MAP kinase inhibiting or modulating activity, and which it is envisaged will be useful in preventing or treating disease states or conditions mediated by the p38 MAP kinases.

Accordingly, in a first aspect, the invention provides the use of a compound for the manufacture of a medicament for the prophylaxis or treatment of a disease state or condition mediated by a p38 MAP kinase; the compound being defined by formula (I): 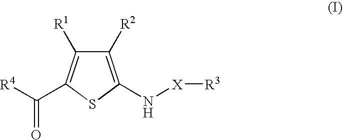
wherein:
R¹ and R² are the same or different and each is selected from hydrogen, C₁₋₄ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members, the aryl and heteroaryl groups each being unsubstituted or substituted by one or more substituent groups R⁷ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}O₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
X¹ is O, S or NR^{c} and X² is ═O, ═S or ═NR^{c};
R^{c} is hydrogen or C₁₋₄ hydrocarbyl;
R⁴ is a group YR⁵ or a group R⁶;
Y is NH, O or S;
R⁵ is selected from (a) carbocyclic and heterocyclic groups having from 3 to 12 ring members; and (b) C₁₋₈ hydrocarbyl groups optionally substituted by one or more substituents selected from hydroxy, oxo, halogen, cyano, amino, mono- or di-C₁₋₄ hydrocarbylamino, and carbocyclic and heterocyclic groups having from 3 to 12 ring members, wherein one or more carbon atoms of the C₁₋₈ hydrocarbyl group may optionally be replaced by O, S, SO, SO₂, NR^{c}, X¹C(X²), C(X²)X¹ or X¹C(X²)X¹, provided that when Y is O, a carbon atom adjacent to the group Y is not replaced by O; and
R⁶ is a heterocyclic group having from 4 to 12 ring members and containing at least one ring nitrogen atom through which R⁶ is linked to the adjacent carbonyl group;
wherein the carbocyclic and heterocyclic groups of substituents R⁵ and R⁶ are each unsubstituted or substituted by one or more substituent groups R⁷ as hereinbefore defined.

Compounds of the formula (I) as defined above have activity in modulating or inhibiting p38 MAP kinase activity. As such, it is anticipated that the compounds possessing such activity will be useful therapeutic agents in the prophylaxis or treatment of diseases where the disease or condition is one in which the activity of p38 MAP kinase initiates or facilitates development of the disease. Examples of conditions ameliorated by the inhibition of p38 MAP kinase are discussed above, and can include, but are not limited to, the said conditions. More particularly, the conditions can be selected from:
(i) inflammatory and arthritic diseases and conditions such as Reiter's syndrome, acute synovitis, rheumatoid arthritis, osteoarthritis, rheumatoid spondylitis, gouty arthritis, traumatic arthritis, rubella arthritis, psoriatic arthritis, graft vs. host reaction and allograft rejections;
(ii) chronic inflammatory lung diseases such as emphysema, chronic pulmonary inflammatory disease, chronic obstructive pulmonary disease (COPD), adult respiratory distress syndrome and acute respiratory distress syndrome (ARDS);
(iii) lung diseases and conditions such as tuberculosis, silicosis, pulmonary sarcoidosis, pulmonary fibrosis and bacterial pneumonia;
(iv) inflammatory diseases and conditions of the enteric tract such as inflammatory bowel disease, Crohn's disease and ulcerative colitis;
(v) toxic shock syndrome and related diseases and conditions such as sepsis, septic shock, endotoxic shock, gram negative sepsis and the inflammatory reaction induced by endotoxin;
(vi) Alzheimer's disease;
(vii) reperfusion injury; and
(vii) diseases and conditions selected from atherosclerosis; muscle degeneration; gout; cerebral malaria; bone resorption diseases; fever and myalgias due to infection, such as influenza; cachexia, in particular cachexia secondary to infection or malignancy, cachexia secondary to acquired immune deficiency syndrome (AIDS); AIDS; ARC (AIDS related complex); keloid formation; scar tissue formation; pyresis and asthma.

Of particular interest are compounds for use in the treatment or prophylaxis of inflammatory diseases and conditions, rheumatoid arthritis and osteoarthritis.

Also of particular interest are compounds for use in the treatment or prophylaxis of chronic obstructive pulmonary disease (COPD).

In another aspect, the invention provides a method for the prophylaxis or treatment of a disease state or condition of the type hereinbefore defined, which method comprises administering to a subject (e.g. a human subject) in need thereof a compound of the formula (I) as defined herein.

In a further aspect, the invention provides a method for the prophylaxis or treatment of a disease state or condition mediated by a p38 MAP kinase, which method comprises administering to a subject (e.g. a human subject) in need thereof a compound of the formula (I) as defined herein.

The invention also provides a method of inhibiting a p38 MAP kinase, which method comprises contacting the p38 MAP kinase with a kinase-inhibiting compound of the formula (I) as defined herein.

The invention further provides a method of modulating a cellular process by inhibiting the activity of a p38 MAP kinase using a compound of the formula (I) as defined herein, which method comprises bringing the compound of formula (I) into contact with a cellular environment containing the p38 MAP kinase.

Many of the compounds of the invention are novel and, in a further aspect, the invention provides a novel compound of the formula (Ia): 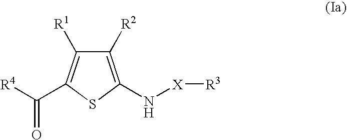
wherein:
R¹ and R² are the same or different and each is selected from hydrogen, C₁₋₄ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members, the aryl and heteroaryl groups each being unsubstituted or substituted by one or more substituent groups R⁷ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
X¹ is O, S or NR^{c} and X² is ═O, ═S or ═NR^{c};
R^{c} is hydrogen or C₁₋₄ hydrocarbyl;
R⁴ is a group YR⁵ or a group R⁶;
Y is NH, O or S;
R⁵ is selected from (a) carbocyclic and heterocyclic groups having from 3 to 12 ring members; and (b) C₁₋₄ hydrocarbyl groups optionally substituted by one or more substituents selected from hydroxy, oxo, halogen, cyano, amino, mono- or di-C₁₋₄ hydrocarbylamino, and carbocyclic and heterocyclic groups having from 3 to 12 ring members, wherein one or more carbon atoms of the C₁₋₈ hydrocarbyl group may optionally be replaced by O, S, SO, SO₂, NR^{c}, X¹C(X²), C(X²)X¹ or X¹C(X²)X¹, provided that when Y is O, a carbon atom adjacent to the group Y is not replaced by O; and
R⁶ is a heterocyclic group having from 4 to 12 ring members and containing at least one ring nitrogen atom through which R⁶ is linked to the adjacent carbonyl group, provided that R⁶ is other than a bicyclic group comprising a benzene ring fused to a 7-membered heterocyclic ring;
wherein the carbocyclic and heterocyclic groups of substituents R⁵ and R⁶ are each unsubstituted or substituted by one or more substituent groups R⁷ as hereinbefore defmind;
provided that:
(a) when X is C═O and R³ is a heteroaryl group substituted by the group R^{a}—R^{b} where R^{a} is NR^{c}C═O, then R^{b} is other than an optionally further substituted phenyl, pyridyl or pyrimidinyl group having a carbocyclic or heterocylic group bonded to the ortho position thereof either directly or through an intervening linker atom or group of 1 or 2 atoms in length;
(b) when X is C═O, R³ is other than:
   (i) an optionally further substituted phenyl, pyridyl or pyrimidinyl group having a carbocyclic or heterocylic group bonded to the ortho position thereof either directly or through an intervening linker atom or group of 1 or 2 atoms in length;
   (ii) a phenyl group having an oxy-substituent bonded to the ortho position thereof;
   (iii) an optionally N-substituted pyrrolidine ring substituted on a carbon atom thereof by a group selected from thiol, substituted thiol, thiocarbonate and groups containing a β-lactam ring;
(c) when X is C═O and R³ is an unsubstituted phenyl group, or a phenyl group substituted by one or more substituents, none of which are cyclic, then R⁴ is other than alkoxy;
(d) when X is C(═O)NH and R³ is a thiophene group bearing a 5-alkoxycarbonyl group, then R⁴ is other than alkoxy;
(e) when Y is NH or O and R⁵ is a C₂₋₄ alkylene group bearing a terminal amino, monoalkylamino or dialkylamino substituent, wherein the alkyl moieties of the mono-and dialkylamino substituents are themselves unsubstituted or further substituted; then X—R³ is other than an unsubstituted or substituted benzoyl group;
(f) when Y is NH and R⁵ is a C₁₋₃ alkylene group bearing a terminal carboxy or alkoxycarbonyl substituent; then X-R³ is other than a 4-carbamimidoyl-benzoyl group;
(g) when X is C═O, Y is NH and R⁵ is a 3-dimethylaminoprop-1-yl group; then R³ is other than a 5-nitro-2-thiophenyl group; and
(h) when X is C═O, R⁴ is ethoxy, R¹ is methyl and R² is hydrogen or cyano; then R³ is other than an unsubstituted phenyl group.

In proviso (b)(ii) of formula (Ia) above, the reference to the “oxy-substituent” means any group in which an oxygen atom of the group is attached directly to the ortho-position of the phenyl ring. Thus, for example, the term includes hydroxy, alkoxy, acyloxy and substituted alkoxy and acyloxy groups.

In proviso (b)(iii) of formula (Ia) above, the reference to “thiocarbonate” means the entity S(C═S)S and includes substituted, unsubstituted and ionised forms of the group. The reference to “groups containing a β-lactam ring” means any group containing either a monocyclic β-lactam ring or a β-lactam ring fused to one or more other rings (as in a penem group).

In a further aspect, the invention provides a compound of the formula (Ia) for use in medicine, for example for use in therapy.

Accordingly, the invention also provides a compound of the formula (Ia) as defined herein for use in the prophylaxis or treatment of a disease state or condition mediated by a p38 MAP kinase.

In another aspect, the invention provides the use of a compound of the formula (Ia) as defined herein for the manufacture of a medicament for the prophylaxis or treatment of a disease state or condition mediated by a p38 MAP kinase.

In this specification, references to formula (I) include formula (Ia) and any sub-group (e.g. formulae II, III, IVa and IVb), example or embodiment of formula (I) and formula (Ia), unless the context indicates otherwise. Thus for example, references to inter alia therapeutic uses, pharmaceutical formulations and processes for making compounds, where they refer to formula (I), are also to be taken as referring to formula (Ia) and any other sub-group of compounds or embodiment of formula (I) and formula (Ia). Similarly, where preferences, embodiments and examples are given for compounds of the formula (I), they are also applicable to compound (Ia) and any sub-groups or embodiments of formula (I) and formula (Ia) unless the context requires otherwise.

In the definition of the compounds of the formula (I) above and as used hereinafter, the term “hydrocarbyl” is a generic term encompassing aliphatic, alicyclic and aromatic groups formed from carbon and hydrogen atoms. In certain cases, as defined herein, one or more of the carbon atoms making up the carbon backbone of the hydrocarbyl group may be replaced by a specified atom or group of atoms. Where stated, the hydrocarbyl groups may be substituted with one or more substituents as defined herein.

Examples of hydrocarbyl groups include saturated groups such as alkyl and cycloalkyl, and groups having varying degrees of unsaturation such as aryl, alkenyl, cycloalkenyl, alkynyl, cycloalkylalkyl, cycloalkenylalkyl, aralkyl, aralkenyl and aralkynyl groups. The examples and preferences expressed below apply to each of the hydrocarbyl substituent groups or hydrocarbyl-containing substituent groups referred to in the various definitions of substituents for compounds of the formula (I) unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alknyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of aryl hydrocarbyl groups include unsubstituted phenyl as well as phenyl substituted by alkyl groups, e.g. toluene, xylene and mesitylene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylnethyl and cyclopentenylmethyl groups.

The term “halogen” as used herein includes fluorine, chlorine, bromine and iodine, but fluorine and chlorine are generally preferred as substituents.

In the general formula (I), the groups R¹ and R² are the same or different and each is selected from hydrogen, C₁₋₄ hydrocarbyl, halogen and cyano.

In one group of compounds of the invention, when R² is cyano, R⁴ is other than C₁₋₆ alkoxy, phenoxy, benzyloxy and C₁₋₆ alkylamino.

In another group of compounds of the invention, R² is selected from hydrogen, C₁₋₄ hydrocarbyl and halogen.

In another embodiment, R¹ is selected from hydrogen, C₁₋₄ hydrocarbyl and halogen.

In a further embodiment, R¹ and R² are the same or different and each is selected from hydrogen, C₁₋₄ hydrocarbyl and halogen.

In general, where R¹ and/or R² is/are halogen, the halogen is preferably selected from chlorine and fluorine, chlorine being particularly preferred.

Where R¹ and/or R² is/are C₁₋₄ hydrocarbyl, the hydrocarbyl group is preferably a saturated hydrocarbyl group, and in particular a C₁₋₃ saturated hydrocarbyl group. Examples of C₁₋₃ saturated hydrocarbyl groups include methyl, ethyl, n-propyl, i-propyl and cyclopropyl. The hydrocarbyl groups are preferably selected from methyl and ethyl, methyl being particularly preferred.

In general, it is preferred that the total number of carbon, halogen and nitrogen atoms making up the substituent groups R¹ and R² does not exceed 5. More particularly, the total number of carbon, halogen and nitrogen atoms making up the substituent groups R¹ and R² is in the range 0 to 4, for example 0, 1, 2 or 3.

Typically, no more than one of the substituent groups R¹ and R² is a halogen.

When a halogen (particularly chlorine) or cyano group is present as one of the groups R¹ and R², the other group is typically hydrogen or methyl.

In one group of compounds of the invention, R¹ is a halogen, preferably chlorine. Particular combinations of groups R¹ and R² include: (a) R¹=chlorine & R²=methyl; (b) R¹=chlorine & R²=hydrogen; (c) R¹=hydrogen & R²=hydrogen;

(d) R¹=methyl & R²=hydrogen; (e) R¹=cyano & R²=methyl; and (f) R¹=methyl & R²=cyano. Presently preferred combinations include combinations (a) and (c).

In the general formula (I), X is selected from C═O, C═S, C(═O)NH, C(═S)NH, C(═O)O, C(═O)S, C(═S)O and C(═S)S.

In a preferred group of compounds of the invention, X is selected from C═O and C(═O)NH.

In one preferred sub-group of compounds, X is C(═O)NH.

In another preferred sub-group of compounds, X is C═O.

In a further group of compounds of the invention, X is selected from C═S, C(═O)NH, C(═S)NH, C(═S)O and C(═S)S.

The group R³ is selected from aryl and heteroaryl groups having from 5 to 12 ring members that can be substituted by one or more groups R⁷. Except where the context indicates otherwise, the term “aryl” as used herein refers to a carbocyclic group having aromatic character, and the term “heteroaryl” refers to a heterocyclic group having aromatic character. The aryl and heteroaryl groups can be monocyclic or bicyclic and can be unsubstituted or substituted with one or more substituents. The terms “aryl” and “heteroaryl” embrace polycyclic (e.g. bicyclic) ring systems wherein one or more rings are non-aromatic, provided that at least one ring is aromatic.

Examples of aryl groups include monocyclic and bicyclic groups containing from six to twelve ring members, and more usually from six to ten ring members. Monocyclic aryl groups are preferred. Particular examples of aryl groups include phenyl, indenyl, tetrahydronaphthyl and naphthyl. The aryl groups may be unsubstituted or substituted with one or more substituents as defined herein.

Examples of heteroaryl groups include monocyclic or bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms, more usually three or fewer, and typically one, two or three. The heteroatoms are typically selected from nitrogen, sulphur and oxygen. In one embodiment, the heteroaryl ring contains at least one ring nitrogen atom. The nitrogen atoms in the heteroaryl rings can be basic, as in the case of a pyridine,or essentially non-basic as in the case of an indole or pyrrole nitrogen. In general the number of basic nitrogen atoms present in the heteroaryl group, including any amino group substituents on the ring, will be less than five.

Examples of heteroaryl groups include but are not limited to pyridine, pyrrole, furan, thiophene, imidazole, oxazole, oxadiazole, oxatriazole, isoxazole, thiazole, isothiazole, pyrazole, pyrazine, pyridazine, pyrimidine, triazine, triazole, tetrazole, quinoline, isoquinoline, benzfuran, benzthiophene, chroman, thiochroman, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole, chromene, isochromene, chroman, isochroman, benzodioxan, quinolizine, benzoxazine, benzodiazine, pyridopyridine, pyrazolopyridine, pyrazolopyrimidine, pyrrolopyridine, pyrrolopyrimidine, quinoxaline, quinazoline, cinnoline, phthalazine, naphthyridine and pteridine groups.

In one general embodiment, X—R³ may be other than a 4-carbamimidoyl-benzoyl group.

It is presently preferred that the group R³ is a monocyclic aryl group or a monocyclic heteroaryl group containing at least one nitrogen atom, for example up to three nitrogen atoms, preferably 0, 1 or 2 nitrogen atoms. Examples of such groups include groups selected from the monocyclic members of the list of specific heteroaryl groups set out above. Examples of groups R³ are phenyl, pyrazolyl, and thiadiazolyl (e.g. [1,3,4]-thiadiazolyl). The groups are optionally substituted by one or more substituent groups R⁷ as defined herein.

Particular examples of groups R³ are as set out in Table 1.

TABLE 1

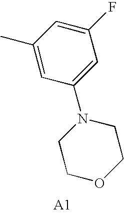

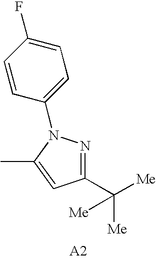

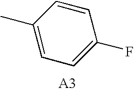

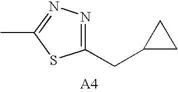

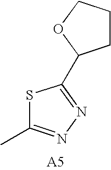

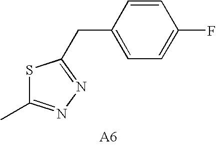

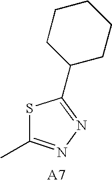

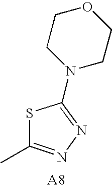

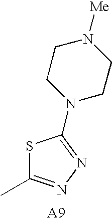

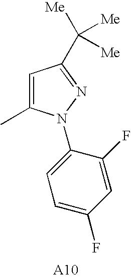

Groups A1, A2 and A10 are particularly preferred.

The group R⁴ is a group YR⁵ or a group R⁶; wherein Y is is NH, O or S; R⁵ is selected from (a) carbocyclic and heterocyclic groups having from 3 to 12 ring members, and (b) optionally substituted C₁₋₈ hydrocarbyl groups; and R⁶ is a heterocyclic group having from 4 to 12 ring members (preferably 4 to 7 ring members), and containing at least one ring nitrogen atom through which R⁶ is linked to the adjacent carbonyl group.

In one preferred group of compounds, Y is NH.

In one general embodiment, when X is C═O and R³ is an unsubstituted or substituted phenyl group, then R⁴ is other than alkoxy

In one embodiment, R⁵ is selected from (a) carbocyclic and heterocyclic groups having from 3 to 12 ring members; and (b) C₁₋₈ hydrocarbyl groups optionally substituted by one or more substituents selected from hydroxy, halogen, cyano, and carbocyclic and heterocyclic groups having from 3 to 12 ring members, wherein one or more carbon atoms of the C₁₋₈ hydrocarbyl group may optionally be replaced by O, S, SO or SO₂ provided that when Y is O, a carbon atom adjacent to the group Y is not replaced by O.

In one preferred embodiment, R⁵ is a carbocylic or heterocyclic group which can be aromatic or non-aromatic. In this embodiment, Y can be NH, O or S, but preferably is NH.

Examples of aromatic carbocyclic and aromatic heterocyclic groups are the aryl and heteroaryl groups defined above in respect of the substituent group R³.

The carbocyclic and heterocyclic groups (e.g. aryl and heteroaryl groups) are preferably monocyclic and typically have from 4 to 7 ring members, more usually 5 or 6 ring members.

When R⁵ is a monocyclic aromatic heterocyclic (heteroaryl) group, one or more nitrogen ring members may be present but it is preferred that no more than three and preferably no more than two nitrogen ring members are present in the group.

Examples of non-aromatic heterocyclic groups include, but are not limited to, rings containing up to three heteroatoms selected from nitrogen, sulphur and oxygen.

Monocylic groups are preferred. Typically at least one nitrogen atom will be present. Particular examples of such groups include piperidine, piperazine, N-methylpiperazine, morpholine, pyrrolidine, imidazoline, imidazolidine, thiazoline, thiazolidine, oxazoline, oxazolidine and tetrahydrofuran. Preferred non-aromatic heterocyclic groups include morpholine and piperidine, particularly morpholine.

Examples of non-aromatic carbocyclic groups include cycloalkyl and cycloalkenyl groups which can be, for example, monocyclic or bicyclic. Particular examples include cycloalkyl and cycloalkenyl groups having from 3 to 10 (e.g. 3 to 7) ring atoms, including groups derived from cyclopropane, cyclobutane, cyclopentane, cyclohexane, cycloheptane, cyclopropene, cyclobutene, cyclopentene, cyclopentadiene, cyclohexene, bicycloheptane, bicyclooctane and decalin.

In another embodiment, R⁵ is a C₁₋₈ hydrocarbyl group optionally substituted by one or more substituents selected from hydroxy, halogen, cyano and carbocyclic and heterocyclic groups having from 3 to 12 ring members. The carbocyclic and heterocyclic groups having from 3 to 12 ring members can be aromatic or non-aromatic groups as defined above in relation to R³, R⁵ and R⁶ and can be unsubstituted or substituted as defined herein.

Where R⁵ is a C₁₋₈ hydrocarbyl group substituted by a carbocyclic or heterocylic group, the hydrocarbyl group can be an alkyl group of up to 4 carbon atoms (more usually up to 3 carbon atoms, for example up to 2 carbon atoms). Examples of such groups include optionally substituted arylmethyl, arylethyl, heteroarylmethyl and heteroarylethyl groups, particular examples being pyridylmethyl and benzyl groups. In one sub-group of compounds of the invention, Y is NH and examples of the YR⁵ moiety include arylmethylamino, arylethylamino, heteroarylmethylamino and heteroarylethylamino groups, particular examples being pyridylmethylamino and benzylamino groups.

In one embodiment, R⁵ is a C₁ hydrocarbyl group substituted by a carbocyclic or heterocyclic group.

In one group of compounds of the invention, one or more (for example 1 or 2) carbon atoms of the C₁₋₈ hydrocarbyl group is replaced by O, S, SO or SO₂ provided that when Y is O, a carbon atom adjacent to the group Y is not replaced by O.

In another group of compounds of the invention, R⁵ is other than a morpholino substituted 1,2,4-triazole group. Alternatively or additionally, R⁵ may be other than an aminocarbonyl substituted alkyl group. In a further alternative, or additionally, when X is C(═O)NH and R³ is a 1,3,4-thiadiazole group, then R⁵ may be other than an alkoxy group.

In another general embodiment, when X is C(═O)NH and R⁴ is YR⁵, then R⁵ may be other than an aryl or heterocyclic ring having attached to an ortho-position thereof a nitrogen-containing linker group bearing a further aryl or heterocyclic group.

In a further general embodiment, when R⁵ is a carbocyclic or heterocyclic group or is a C₁₋₈ hydrocarbyl group substituted by a carbocyclic or heterocyclic group, the said carbocyclic or heterocyclic group may be unsubstituted or substituted only by one or more non-cyclic substituents.

The group R⁶ is a heterocyclic group having from 4 to 12 ring members and containing at least one ring nitrogen atom through which R⁶ is linked to the adjacent carbonyl group. Preferably the heterocyclic group is monocyclic and has 4 to 7 ring members, more typically 5 to 7 ring members and more preferably 5 or 6 ring members. Six membered heterocyclic rings are particularly preferred. The heterocyclic group may be aromatic (for example a pyrrole or substituted pyrrole group), but more usually is non-aromatic, and preferably is saturated. The heterocyclic group typically contains up to 4 heteroatom ring members, more usually up to 3, for example 1 or 2. The heteroatom ring members are typically chosen from nitrogen, oxygen and sulphur, with nitrogen and oxygen being preferred. The group R⁶ can be unsubstituted or substituted by one or more subsitutents R⁷ as hereinbefore defined. In one embodiment, the group R⁶ is unsubstituted or is substituted by one or more substituents selected from oxo; halogen; hydroxy; cyano; C₁₋₂ saturated hydrocarbyloxy optionally substituted by hydroxy, methoxy, oxo, halogen or cyano; and C₁₋₃ saturated hydrocarbyl optionally substituted by hydroxy, methoxy, oxo, halogen or cyano. Preferably the group R⁶ is unsubstituted or is substituted by one or more methyl, ethyl and hydroxymethyl.

Examples of substituent groups R⁶ include the non-aromatic nitrogen-containing heterocyclic groups defined above in relation to the group R⁵. Particular examples of substituent groups R⁶ include unsubstituted or substituted piperidine, piperazine, N-methylpiperazine, morpholine, pyrrolidine, imidazoline, imidazolidine, thiazolidine and oxazolidine groups. Presently preferred groups R⁶ include unsubstituted or substituted morpholine, piperidine, piperazine and N-methyl piperazine groups, with morpholine being particularly preferred.

In one preferred embodiment, R⁶ is a group: 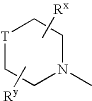
where T is N-methyl or O; R^{x} and R^{y} are the same or different and are selected from hydrogen and methyl; or one of R^{x} and R^{y} is selected from hydroxymethyl and ethyl and the other is hydrogen. Preferably T is O and R^{x} and R^{y} are both hydrogen.

Where reference is made herein to carbocyclic and heterocyclic groups, and aryl and heteroaryl groups, the said groups can each be unsubstituted or substituted by one or more substituent groups R⁷ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}R^{d}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; where X¹, X² and R^{c} are as hereinbefore defined and R^{d} is hydrogen or C₁₋₄ hydrocarbyl.

Where the substituent group R⁷ comprises or includes a carbocyclic or heterocyclic group, the said carbocyclic or heterocyclic group may be unsubstituted or may itself be substituted with one or more further substituent groups R⁷. In one sub-group of compounds of the formula (I), such further substituent groups R⁷ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R⁷.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

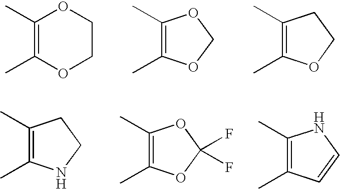

When R^{b} is a C₁₋₈ hydrocarbyl group, one or more carbon atoms of a hydrocarbyl group may optionally be replaced by O, S, SO, SO₂, NR^{c}, X¹C(X²), C(X²)X¹ or X¹C(X²)X¹ wherein X¹ and X² are as hereinbefore defined. For example, 1, 2, 3 or 4 carbon atoms of the hydrocarbyl group may be replaced by one of the atoms or groups listed provided that at least one carbon atom is present in the hydrocarbyl group, and the replacing atoms or groups may be the same or different. Examples of groups in which a carbon atom of the hydrocarbyl group has been replaced by a replacement atom or group as defined above include ethers and thioethers (C replaced by O or S), amides, esters, thioamides and thioesters (C replaced by X¹C(X²) or C(X²)X¹), sulphones and sulphoxides (C replaced by SO or SO₂) and amines (C replaced by NR^{c}).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}—R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

In one sub-group of compounds of the invention, the substituent R³ is a monocyclic aryl or heteroaryl group of 5 or 6 ring members wherein the aryl or heteroaryl group bears a substituent group which is a 4-7 membered carbocylic and heterocyclic group. The carbocyclic or heterocyclic substituent can be linked to the aryl or heteroaryl group via a carbon-nitrogen bond.

The carbon atom of the carbon-nitrogen bond can form part of the aryl or heteroaryl group, or the carbon atom of the carbon-nitrogen bond can form part of the substituent group.

When the carbon atom of the carbon-nitrogen bond forms part of the substituent group, the substituent group can be for example an optionally substituted phenyl ring attached to the heteroaryl group via a nitrogen atom in the heteroaryl group. The optional substituents on the phenyl ring may be selected from the list set out above in relation to R⁷. A preferred substituent is fluoro, for example para-fluoro.

When the nitrogen atom of the carbon-nitrogen bond forms part of the substituent group, the substituent group can be, for example, a 4 to 7 membered (more typically 5 to 6 membered) heterocyclic group R⁸ containing at least one nitrogen atom. Preferred heterocyclic groups in this context include morpholino, piperidino, piperazino, N-methyl piperazino and pyrrolidino, with morpholino being particularly preferred.

Where the group R³ is a phenyl group, it can be optionally substituted by one or more substituents R⁷ as hereinbefore defined. One sub-group of compounds is the group of compounds wherein the phenyl ring contains one or two meta substituents, for example wherein one meta position on the phenyl ring is unsubstituted or is substituted by a group selected from fluorine, chorine, methoxy, trifluoromethoxy, trifluoromethyl, ethyl, methyl and isopropyl; and the other meta position is substituted by a group selected from fluorine, chorine, methoxy, trifluoromethoxy, trifluoromethyl, ethyl, methyl, isopropyl, isobutyl, t-butyl, phenyl, substituted phenyl, and five and six membered monocyclic heterocyclic groups.

One particular combination of meta substituents is the combination of a halogen, preferably fluoro, and a group R⁸ as hereinbefore defined.

Where the group R³ is a heteroaryl group, it can be, for example, a pyrazole group optionally substituted by one or more substituents R⁷ as hereinbefore defined. The pyrazole group can have, for example, one or two such substituent groups R⁷. Where there are two substituent groups R⁷ present, it is preferred that they are located on non-adjacent ring members. It is further preferred that at least one of the substituents is located at a position meta or β with respect to the ring member linked to the group X.

One particularly preferred group of compounds is the group wherein the heteroaryl group R³ is a pyrazolyl ring substituted by an optionally substituted phenyl group (e.g. 4-fluorophenyl) and a C₁₋₄ hydrocarbyl group, e.g. a tert-butyl group or a tert-butyl isostere.

Another particularly preferred group of compounds is the group wherein the heteroaryl group R³ is a thiadiazole group (e.g. a [1,3,4]-thiadiazole group).

One group of compounds of the invention is defined by the general formula (II); 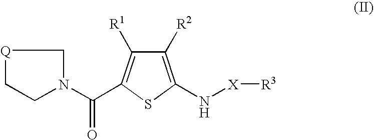
wherein R¹, R² and R³ are as hereinbefore defined, and Q is selected from CH₂, OCH₂, NHCH₂, N(CH₃)CH₂ or CH₂CH₂. A preferred group Q is OCH₂.

Another group of compounds of the invention is represented by the formula (III):

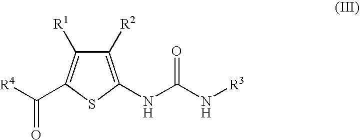

wherein R¹ to R⁴ are as hereinbefore defined.

Within the group of compounds of the formula (III) are compounds of the formula (IVa): 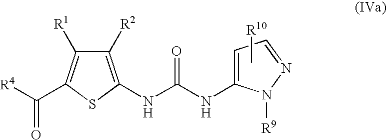
wherein R¹, R² and R⁴ are as hereinbefore defined;
R⁹ is selected from carbocyclic and heterocyclic groups having from 3 to 7 ring members; a group R^{e}—R^{f} wherein R^{e} is a bond, CO, X¹C(X²), C(X²)X¹, X¹C(X²)X¹, SO, SO₂, SO₂NR^{c} or NR^{c}SO₂; and R^{f} is selected from (a) hydrogen, (b) carbocyclic and heterocyclic groups having from 3 to 7 ring members, and (c) a C₁₋₈ hydrocarbyl group optionally substituted by one or more substituents selected from hydroxy, oxo, halogen, cyano, nitro, amino, mono- or di-C₁₋₄ hydrocarbylamino, and carbocyclic and heterocyclic groups having from 3 to 7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; where X¹, X² and R^{c} are as hereinbefore defined; and
R¹⁰ is selected from hydrogen, halogen and C₁₋₆ hydrocarbyl optionally substituted by one or more substituents selected from hydroxy, oxo, halogen, cyano, nitro, and wherein one or more carbon atoms of the C₁₋₆ hydrocarbyl group may optionally be replaced by O, S, SO, SO₂, NR^{c}, X¹C(X²), C(X²)X¹ or X¹C(X²)X¹; where X¹, X² and R^{c} are as hereinbefore defined.

In the group of compounds defined by formula (IVa), R⁹ is preferably a phenyl group, for example a fluorophenyl group (e.g. a 4-fluorophenyl group); and R¹⁰ is preferably a hydrogen atom or a C₁₋₆ alkyl group, particular examples of which are methyl, ethyl, propyl, isopropyl, butyl, isobutyl and tertiary butyl; with tertiary butyl being particularly preferred.

A further group of compounds within the general formula (III) is the group of compounds of the formula (IVb):

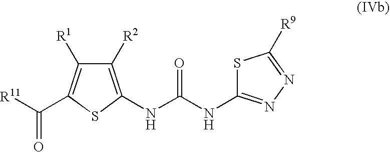

wherein R¹¹ is R⁶ or NHR⁵; and R¹, R², R⁵, R⁶ and R⁹ are as hereinbefore defined.

For the avoidance of doubt, it is to be understood that each general and specific preference, embodiment and example of any one group selected from R¹, R², R³ R⁴, R⁵, R⁶, R⁷, R⁸, R⁹ and R¹⁰ and sub-groups thereof may be combined with each general and specific preference, embodiment and example of any one or more other groups selected from R¹, R², R³ R⁴, R⁵, R⁶, R⁷, R⁸, R⁹ and R¹⁰ and sub-groups thereof and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Specific examples of novel compounds within the scope of the present invention include:
3-chloro-5-(3-fluoro-5-morpholin-4-yl-benzoylamino)-4-methyl-thiophene-2-carboxylic acid methyl ester;
N-[4-chloro-3-methyl-5-(morpholine-4-carbonyl)-thiophen-2-yl]-3-fluoro-5-morpholin-4-yl-benzamide;
5-{3-[5-tert-butyl-2-(4-fluoro-phenyl)-2H-pyrazol-3-yl]-ureido}-3-chloro-4-methyl-thiophene-2-carboxylic acid methyl ester;
1-[5-tert-butyl-2-(4-fluoro-phenyl)-2H-pyrazol-3-yl]-3-[4-chloro-3-methyl-5-(morpholine-4-carbonyl)-thiophen-2-yl]-urea;
5-(3-fluoro-5-morpholin-4-yl-benzoylamino)-3-methyl-thiophene-2-carboxylic acid ethyl ester;
3-fluoro-N-[4-methyl-5-(morpholin-4-carbonyl)-thiophen-2-yl]-5-morpholin-4-yl-benzamide;
5-{3-[5-tert-butyl-2-(4-fluoro-phenyl)-2H-pyrazol-3-yl]-ureido}-thiophene-2-carboxylic acid ethyl ester;
1-[5-tert-butyl-2-(4-fluorophenyl)-2H-pyrazol-3-yl]-3-[5-(morpholine-4-carbonyl)-thiophen-2-yl]-urea;
5-{3-[5-tert-butyl-2-(4-fluoro-phenyl)-2H-pyrazol-3-yl]-ureido}-3-methyl-4-4-cyano-thiophene-2-carboxylic acid methyl ester;
3-cyano-5-(4-fluorobenzoylamino)-4-methyl-thiophene-2-carboxylic acid methyl ester;
N-[4-chloro-3-methyl-5-(morpholine-4-carbonyl)-thiophen-2-yl]-4-fluorobenzamide;
N-[4-chloro-3-methyl-5-(4-fluoro-phenylaminocarbonyl)-thiophen-2-yl]-4-fluorobenzamide;
3-chloro-5-(4-fluorobenzoylamino)-4-methyl-thiophene-2-carboxylic acid methyl ester;
1-[5-tert-butyl-2-(4-fluoro-phenyl)-2H-pyrazol-3-yl]-3-[4-chloro-3-methyl-5-(1-methylpiperazine-4-carbonyl)-thiophen-2-yl]-urea;
1-[5-tert-butyl-2-(4-fluoro-phenyl)-2H-pyrazol-3-yl]-3-[4-chloro-3-methyl-5-(4-pyridylmethylaminocarbonyl)-thiophen-2-yl]-urea;
N-[4-chloro-3-methyl-5-(4-pyridylmethylaminocarbonyl)-thiophen-2-yl]-3-fluoro-5-morpholin-4-yl-benzamide;
N-[4-chloro-3-methyl-5-(2,3,5-trimethyl-2H-pyrazol-4-ylaminocarbonyl)-thiophen-2-yl]-3-fluoro-5-morpholin-4-yl-benzamide;
N-[4-chloro-3-methyl-5-(4-fluorophenylaminocarbonyl)-thiophen-2-yl]-3-fluoro-5-morpholin-4-yl-benzamide;
N-[4-chloro-3-methyl-5-(1-methylpiperazin-4-ylaminocarbonyl)-thiophen-2-yl]-3-fluoro-5-morpholin-4-yl-benzamide;
N-[4-chloro-3-methyl-5-(2-amino-pyrimidin-5-ylaminocarbonyl)-thiophen-2-yl]-3-fluoro-5-morpholin-4-yl-benzamide;
1-[2-(tetrahydrofuran-2-yl)-thiadiazol-5-yl]-3-[4-chloro-3-methyl-5-(morpholine-4-carbonyl)-thiophen-2-yl]-urea;
1-[4-chloro-3-methyl-5-(morpholine-4-carbonyl)-thiophen-2-yl]-3-[5-cyclohexyl-[1,3,4]thiadiazol-2-yl]-urea;
1-[4-chloro-3-methyl-5-(morpholine-4-carbonyl)-thiophen-2-yl]-3-(5-morpholin-4-yl-[1,3,4]thiadiazol-2-yl)-urea;
1-[4-chloro-3-methyl-5-(morpholine-4-carbonyl)-thiophen-2-yl]-3-[5-(4-methyl-piperazin-1-yl)-[1,3,4]thiadiazol-2-yl]-urea; and
1-[5-tert-Butyl-2-(2,4-difluoro-phenyl)-2H-pyrazol-3-yl]-3-[4-chloro-3-methyl-(morpholine-4-carbonyl)-thiophen-2-yl]-urea.

In a further aspect, the invention provides compounds of the formula (I) as hereinbefore defined for use in medicine and pharmaceutical compositions comprising a compound of the formula (I) in association with a pharmaceutically acceptable carrier.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH⁴⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:
C₁₋₇ alkyl (e.g., Me, Et, n-Pr, i-Pr, n-u, s-Bu, i-Bu, t-Bu);
C₁₋₇ aminoalkyl (e.g., aminoethyl; 2-(N,N-diethylamino)ethyl;
2-(4-morpholino)ethyl); and
acyloxy-C₁₋₇ alkyl (e.g., acyloxymethyl; acyloxyethyl; pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)-carbonyloxyethyl; 4-tetrahydropyranyl)carbonyloxymethyl; and 1-(4-tetrahydro-pyranyl)carbonyloxyethyl).

Also, some prodrugs are activated enzymatically to yield the active compound, or a compound which, upon further chemical reaction, yields the active compound (for example, as in Antibody-directed Enzyme Prodrug Therapy (ADEPT), Gene-directed Enzyme Prodrug Therapy (GDEPT), Polymer-directed Enzyme Prodrug Therapy (PDEPT), Ligand-directed Enzyme Prodrug Therapy (LIDEPT), etc.). For example, the prodrug may be a sugar derivative or other glycoside conjugate, or may be an amino acid ester derivative.

Where the compounds of the formula (I) contain chiral centres, all individual optical forms such as enantiomers, epimers and diastereoisomers, as well as racemic mixtures of the compounds are within the scope of formula (I).

Methods for the Preparation of Compounds of the Formula (I)

Compounds of the formula (I) can be prepared by reacting a compound of the formula (V): 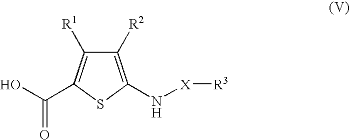
or an activated derivative thereof, with an amine, thiol or hydroxyl compound suitable for introducing the residue YR⁵ or R⁶. For example, where R⁴ is a group R⁶ such as a morpholino group containing a nitrogen atom, or is a group NHR⁵, the corresponding amine R⁶H or R⁵NH₂ can be reacted with the compound of the formula (V).

The coupling reaction between the amine and the carboxylic acid (V) can be carried out by forming an activated derivative of the acid such as an acid chloride (e.g. by reaction with thionyl chloride), and then reacting the acid chloride with the amine, for example by the method described in Zh. Obs. Khini. 31, 201 (1961), and the method described in U.S. Pat. No. 3,705,175. Alternatively, acid chlorides can be formed by reacting the acid with oxalyl chloride the presence of dimethyl formamide, or by forming the carboxylate salt and reacting the salt with oxalyl chloride.

Alternatively, and more preferably, the coupling reaction between the carboxylic acid (XII) and the morpholine compound (XIII) can be carried out in the presence of an amide coupling reagent of the type commonly used to form peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (EDAC) (Sheehan et al, J. Org. Chem., 1961, 26, 2525), uronium-based coupling agents such as O-(7-azabenzotriazol-1-yl)-N,N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC and DCC in combination with HOAt or HOBt.

The coupling reaction is typically carried out in a non-aqueous, non-protic solvent such as dimethylsulfoxide, dichloromethane, dimethylformamide or N-methylpyrrolidine. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

Compounds of the formula (V) can be prepared by hydrolysis of a compound of the formula (I) in which R⁴ is a methoxy group, i.e. a compound of the formula (VI):

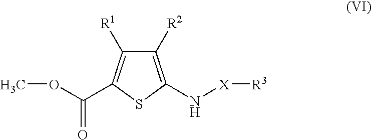

wherein R¹ to R³ are as hereinbefore defined. The hydrolysis reaction can be effected using standard methods, for example by treatment with an alkali metal hydroxide such as lithium hydroxide. The reaction is typically carried out in an aqueous solvent, optionally in the presence of a miscible co-solvent such as methanol or ethanol with heating to a non-extreme temperature between room temperature and 100° C., preferably a temperature below 80° C.

Compounds of the formula (VI) in which X is CO can be prepared from compounds of the formula (VII):

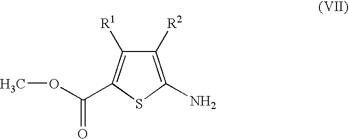

by reaction with a compound of the formula R³COOH or an activated derivative thereof such as an acid chloride in accordance with standard methods. Thus, for example, an acid chloride can be generated using oxalyl chloride and dimethylformamide in a non-protic solvent such as dichloromethane. Alternatively, coupling of the amine and carboxylic acid can be effected using one or more of the peptide coupling reagents described above.

Compounds of the formula (VI) in which X is CONH, C(O)O and C(O)S can be prepared by reaction of a compound of the formula (VII) with a compound of the formula R³NH₂, R³OH, or R³SH and phosgene. The reaction is typically carried out in a non protic solvent such as dichloromethane or toluene, for example at a moderate temperature such as room temperature.

Compounds of the formula (VI) in which X is C(═S)NH can be prepared by reacting a compound of the formula (VII) with an isothiocyanate R³NCS according to standard methods. Compounds of the formula (VI) in which X is C(═S), C(═S)NH, C(═S)O and C(═S)S can be prepared from compounds of the formula (VIII):

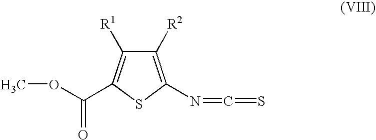

by reaction with a compound of the formula R³NH2, R³O or R³SO in accordance with standard methods. Examples of such methods can be found in Synthesis, Vol. 1, pp 108-118 (2001), Heterocyclic Chemistry, Vol. 17(8), pp 1789-92 (1980) and Zh. Org. Khim. Vol. 12(7), pp 1532-1535 (1976).

Compounds of the formula (VIII) can be prepared from the corresponding amine (VII) by reaction with thiophosgene, for example as described in Kryczka et al., Organiki, pp 65-72, 2001 and Grayson, Organic Process Research & Development, Vol.1(3), pp 240-246 (1997).

Compounds of the formula (VII) are commercially available or can be prepared by nitration and reduction of a compound of the formula (IX):

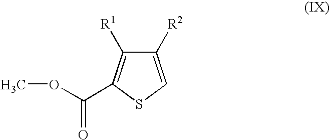

Nitration of the compound of the formula (IX) can be achieved using standard conditions well known to the skilled chemist. For example, the compound of the formula (IX) can be reacted with acetic acid and nitric acid in acetic anhydride, in the presence of a co-solvent, e.g. a halogenated hydrocarbon such as dichloromethane. Where required, the reaction mixture may be heated, for example to a temperature of up to about 100° C., more preferably up to about 80° C.

The resulting nitro-intermediate is reduced to give the amine using a suitable reducing agent. Thus, for example, reduction can be effected using a mixture of powdered iron and iron sulphate in an aqueous solvent optionally containing a water-miscible co-solvent such as dioxane.

Compounds of the formula (I) in which X is C(═O)NH can be prepared by reacting a compound of the formula (X): 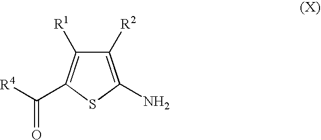
with phosgene and subsequently with a compound of the formula R³NH₂. The reaction is typically carried out in a dry aprotic solvent such as dichloromethane at a non-extreme temperature, for example at room temperature.

Compounds of the formula (X) can be prepared by nitration of a compound of the formula (XI) and subsequent reduction of the nitro group to an amino group.

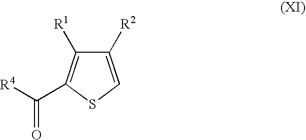

Nitration can be carried out using nitration conditions known to be suitable for nitrating thiophenes. For example, nitration may be effected using a nitronium salt such as nitronium tetrafluoroborate in a polar aprotic solvent such as acetonitrile. The reaction is typically carried out ambient temperatures or lower.

The compounds of the formula (XI) can be prepared by reacting a carboxylic acid of the formula (XII) with an agent suitable for introducing the group R⁴.

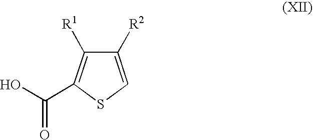

For example, when R⁴ is a cyclic amino group R⁶, or an amino group of the formula NHR⁵, the carboxylic acid of the formula (XII) can be reacted with the appropriate amine using methods described above. For example, an acid chloride may be prepared from the acid and then reacted with the amine. Alternatively, and more preferably, a peptide coupling reagent such as HOAt and HOBT may be used as described above.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). A hydroxy group may be protected, for example, as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NR^{c}O—R) or a urethane (—NR^{c}O—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulfonyl (tosyl) and methanesulfonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetarnidomethyl ether (—S—CH₂NHC(═O)CH₃).

Novel Chemical Intermediates

Many of the intermediate compounds (in particular compounds of the formula (V) above) used in the synthesis of the compounds of the formula (I) are novel and, as such, represent a further aspect of the invention.

Examples of particular groups of intermediate compounds believed to be novel include compounds of the formulae (X) and (XI):

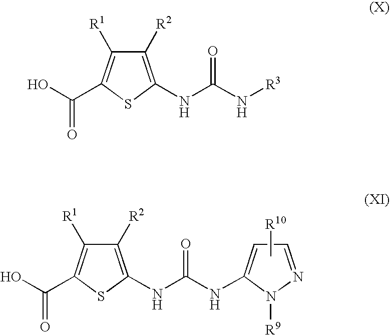

wherein R¹, R², R³, R⁹ and R¹⁰ are as hereinbefore defined.

Pharmaceutical Formulations

The invention also provides compounds of the formula (I) as hereinbefore defined in the form of pharmaceutical compositions.

The pharmaceutical compositions can be in any form suitable for oral, parenteral, topical, intranasal, intra-articular, ophthalmic, otic, rectal, intra-vaginal, or transdermal administration, or administration by inhalation. Where the compositions are intended for parenteral administration, they can be formulated for intravenous, intramuscular or subcutaneous administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a celluloses or derivative thereof such as methyl cellulose, ethyl cellulose, hydroxypropyl methyl cellulose, and starches such as corn starch. Tablets may also contain such standard ingredients as binding and granulating agents agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g. tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral or intra-articular administration are typically presented as sterile aqueous or oily solutions or fine suspensions, or may be provided in finely divided sterile powder form for making up extemporaneously with sterile water for injection.

Examples of formulations for rectal or intra-vaginal administration include foams, or pessaries and suppositories which may be, for example, formed from a shaped mouldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams.

The active compound will be administered to a patient in need thereof (for example a human or animal patient) in an amount sufficient to achieve the desired therapeutic effect.

Methods of Treatment

It is envisaged that the compounds of the formula (I) will useful in the prophylaxis or treatment of a range of disease states or conditions mediated by p38 MAP kinases. Examples of such disease states and conditions are set out above.

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

A typical daily dose of the compound can be in the range from 100 picograms to 10 milligrams per kilogram of body weight, more typically 10 nanograms to 1 milligram per kilogram of bodyweight although higher or lower doses may be administered where required. Ultimately, the quantity of compound administered will be commensurate with the nature of the disease or physiological condition being treated and will be at the discretion of the physician.

The compounds of the formula (I) can be administered as the sole therapeutic agent or they can be administered in combination therapy with one or more other compounds for treatment of a particular disease state, for example rheumatoid arthritis, osteoarthritis, chronic lung inflammatory diseases (e.g. COPD) and inflammatory bowel diseases. Examples of other therapeutic agents that may be administered together (whether concurrently or at different time intervals) with the compounds of the formula (I) include methotrexate, prednisilone, sulfasalazine, leflunomide and NSAIDs, for example COX-2 inhibitors such as celecoxib, rofecoxib, valdecoxib and lumiracoxib, bronchodilators, e.g. beta agonists and anticholinergics such as salbutamol, salmeterol and ipatropium bromide; corticosteroids such as fluticasone proprionate; mucolytics such as guaifenesin; and antibiotics.

### EXAMPLES

The invention will now be illustrated, but not limited, by reference to the specific embodiments described in the following examples.

In the examples, the compounds prepared were characterised by liquid chromatography and mass spectroscopy using two systems, the details of which are set out below. The two systems were equipped with identical chromatography columns and were set up to run under the same operating conditions. The operating conditions used are also described below.

1. Platform System

System: Waters 2790/ Platform LC
Mass Spec Detector: Micromass Platform LC
PDA Detector: Waters 996 PDA
   Analytical Conditions:
Eluent A: H₂O (1% Formic Acid)
Eluent B: CH₃CN (1% Formic Acid)
Gradient: 5-95% eluent B
Flow: 1.5 ml/min
Column: Synergi 4 μm Max-RP C₁₂, 80A, 50×4.6 mm (Phenomenex)
   MS Conditions:
Capillary voltage: 3.5 kV
Cone voltage: 30 V
Source Temperature: 120
   2. FractionLynx System
System: Waters FractionLynx (dual analytical/prep)
Mass Spec Detector: Waters-Micromass ZQ
PDA Detector: Waters 2996 PDA
   Analytical Conditions:
Eluent A: H₂O (1% Formic Acid)
Eluent B: CH₃CN (1% Formic Acid)
Gradient: 5-95% eluent B
Flow: 1.5 ml/min
Column: Synergi 4 μm Max-RP C₁₂, 80A, 50×4.6 mm (Phenomenex)
   MS Conditions:
Capillary voltage: 3.5 kV
Cone voltage: 30 V
Source Temperature: 120
Desolvation Temperature: 230

The starting materials for each of the Examples are commercially available unless otherwise specified.

### Example 1

### 1A. Preparation of 3-fluoro-5-morpholin-4-yl-benzoic acid

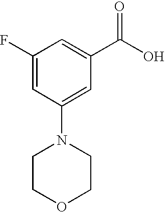

To a solution of 3,5-di-fluorobenzoic acid (commercially available from Aldrich) (10 g, 63.3 mmol) in ethanol (100 ml) was added concentrated sulphuric acid (5 ml) and the reaction was heated at 80° C. for 48 hours. The reaction mixture was evaporated and the residue was partitioned between ethyl acetate and 2N sodium hydroxide. The organic layer was washed with saturated brine solution, dried (MgSO₄), filtered and evaporated to afford 3,5-di-fluorobenzoic acid ethyl ester as a pale yellow oil (8.79 g) which was used immediately in the next step without purification; δ_{H} (400 Mz, CDCl₃) 7.6 (m, 2H), 7.0 (m, 1H), 4.4 (q, 2H), 1.4 (t, 3H).

A mixture of 3,5-di-fluorobenzoic acid ethyl ester (8.79 g, 47.5 mmol) and morpholine (20 ml) in dimethylsulphoxide (250 ml) was heated at 100° C. with stirring for 3 days. The reaction was cooled and then partitioned between diethyl ether and water. The aqueous layer was extracted several times with diethyl ether and the organics were combined and dried over MgSO₄ before filtering the solution and evaporating the solvent under reduced pressure. The residue was subjected to purification by flash chromatography on silica gel. Eluting with 1:4 ethyl acetate:petroleum ether afforded 3-fluoro-5-morpholin-4-yl-benzoic acid ethyl ester as a yellow oil (4.8 g); δ_{H} (400 MHz, CDCl₃) 7.4 (s, 1H), 7.2 (d, 1H), 6.8 (d, 1H), 4.4 (q, 2H), 3.8 (t, 4H), 3.2 (t, 4H), 1.4 (t, 3H).

A solution of 3-fluoro-5-morpholin-4-yl-benzoic acid ethyl ester (4.8 g, 18.9 mmol) in ethanol (20 ml) was treated with 2N sodium hydroxide (20 ml) and the reaction mixture was stirred at room temperature overnight. The reaction mixture was evaporated under reduced pressure and the residue was partitioned between ethyl acetate and water. The aqueous layer was acidified with 2N HCl and the solid precipitate was filtered, washed with diethyl ether and then dried to give the title compound as a white solid (3.1 g). LC MS—M+H 226

### 1B. Preparation of 3-chloro-4-methyl-5-aminothiophene-2-carboxylic acid methyl ester

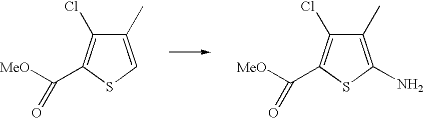

To a solution of 3-chloro-4-methyl-thiophene-2-carboxylic acid methyl ester (9 g, 47.37 mmol) in acetic anhydride (50 ml) and dichloromethane (70 ml) was added a mixture of acetic acid and concentrated nitric acid (5:1, 60 ml) at room temperature. The resulting solution was then heated to 80° C. for a period of 24 hours. Upon cooling, the solvent was removed under reduced pressure and the residue was dissolved in dichloromethane (250 ml). The organic solution was washed with saturated sodium bicarbonate solution (50 ml) and brine (50 ml) before drying over MgSO₄. The resulting solution was filtered and the solvent was removed under reduced pressure to afford the crude product (12.9 g) which was used immediately in the next step without purification.

To a solution of the crude 3-chloro-4-methyl-5-nitrothiophene-2-carboxylic acid methyl ester (12.9 g, 54.9 mmol) in dioxane (250 ml) and water (50 ml) was added iron powder (27.6 g, 0.494 mol) followed by iron sulphate heptahydrate (33.6 g, 0.121 mol). The reaction mixture was then heated to reflux for 4 hours before cooling to room temperature. The solvent was then removed under reduced pressure and the residue was partitioned between ethyl acetate (150 ml) and 1N HCl (100 ml). The organic layer was separated and the aqueous layer was then basified with saturated sodium bicarbonate solution. The solution was extracted with ethyl acetate (2×250 ml), the organic layers were combined, dried (MgSO₄), filtered and the solvent was removed under reduced pressure. The residue was subjected to purification by flash column chromatography on silica gel, eluting with 15% ethyl acetate/petroleum ether to afford the title compound as an off white crystalline solid (1.77 g, 18% over two steps); LC MS M+H 206

### 1C. 3-Chloro-5-(3-Fluoro-5-morpholin-4-yl-benzoylamino)-4-methyl-thiophene-2-carboxylic acid methyl ester

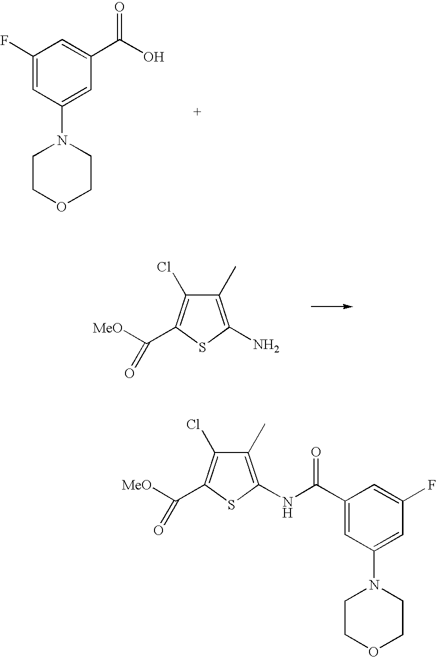

To a solution of 3-morpholino-5-fluorobeiizoic acid (2.42 g, 10.75 mmol) in dichloromethane (100 ml) was added oxalyl chloride (1.11 ml, 12.90 mmol) followed by dimethylformamide (2 drops). The resulting solution was then stirred at room temperature, under an atmosphere of nitrogen, for a period of 4 hours. The solvent was then removed under reduced pressure and the residue was azeotroped to dryness by co-evaporation with toluene (2×50 ml). The resulting solid was then dissolved in dichloromethane (100 ml) and to the solution was added diisopropylethylamine (5.62 ml, 32.19 mmol) followed by cautious addition of the aminothiophene product of Example 1B (2.2 g, 10.73 mmol). After stirring at room temperature under nitrogen for 17 hours, the reaction mixture was diluted with dichloromethane (150 ml) and partitioned with 1N HCl (50 ml). The organic layer was separated, washed successively with saturated sodium bicarbonate solution (50 ml) and brine (50 ml), dried (MgSO₄), filtered and concentrated. Purification by flash chromatography eluting with ethyl acetate/petroleum ether (1:4) gave the title compound as a white crystalline solid (1.60 g, 36%); LC MS M+H 413

### 1D. 3-Chloro-5-(3-Fluoro-5-morpholin-4-yl-benzoylamino)-4-methyl-thiophene-2-carboxylic acid

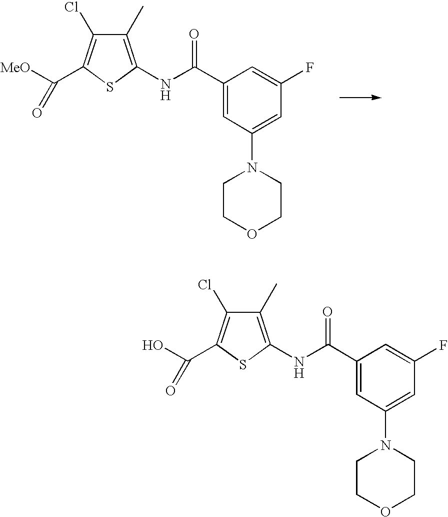

To a suspension of the ester product of Example 1C (0.903 g, 1.9 mmol) in methanol:water [2:1] (30 mls) was added lithium hydroxide (0.33 g, 7.6 mmol) and the reaction was heated at 60° C. overnight. The solution was evaporated under reduced pressure and the residue was partitioned between ethyl acetate and water. The aqueous layer was acidified and extracted with ethyl acetate, dried (MgSO₄), filtered and evaporated under reduced pressure to give the crude title compound as an orange foam (0.6 g); LC MS M+H 399.

### 1E. Preparation of N-[4-Chloro-3-methyl-5-(morpholine-4-carbonyl)-thiophen-2-yl]-3-fluoro-5-morpholin4-yl-benzamide

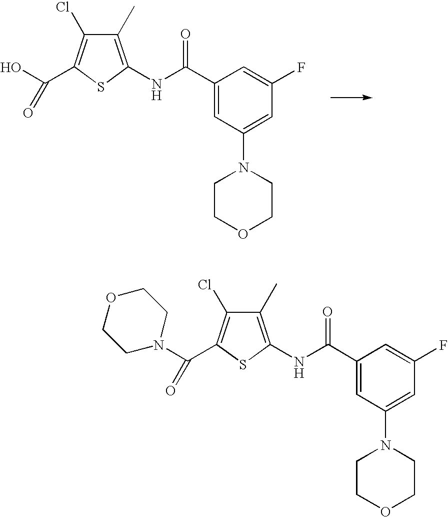

To a solution of the product of Example 1D, 3-chloro-5-(3-fluoro-5-morpholin-4-yl-benzoylamino)-4-methyl-thiophene-2-carboxylic acid, (100 mg, 0.25 mmol ) in dimethylsulphoxide (2 ml) was added EDAC (72 mgs, 0.37 mmol), HOAt (50 mgs, 0.37 mmol) followed by morpholine (22 mgs, 0.25 mmol). The reaction mixture was stirred at room temperature overnight, and the resultant solid was filtered and washed with methanol, affording the title product as an off-white solid (40 mg). LC MS M+H 469

### Example 2

### 2A. Preparation of 5-{3-[5-tert-butyl-2-(4-fluoro-phenyl)-2H-pyrazol-3-yl]-ureido}-3-chloro-4-methyl-thiophene-2-carboxylic acid methyl ester

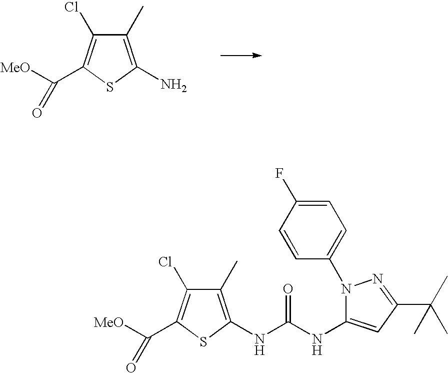

To a solution of the product of Example 1B, 3-chloro-4-methyl-5-aminothiophene-2-carboxylic acid methyl ester (see Example 1B) (0.8 g, 3.9 mmol) in dichloromethane (70 ml) was added 20% phosgene in toluene (7.73 ml) and the reaction mixture was stirred at room temperature overnight. Excess phosgene was then blown off using nitrogen gas over a period of 30 minutes. 5-tert-Butyl-2-(4-fluoro-phenyl)-2H-pyrazol-3-ylamine (0.9 g, 3.9 mmol) was added in one portion and the reaction mixture was stirred at room temperature for 48 hours. Methanol (10 ml) was added, the reaction mixture was stirred for 30 mins then diluted with dichloromethane, washed with saturated bicarbonate and saturated brine solution. The organic layers were dried (MgSO₄), filtered and evaporated to give a brown oil. The residue was subjected to purification by flash chromatography on silica gel. Eluting with 1:4 ethyl acetate: petroleum ether afforded the title compound (0.5 g); LC MS M+H 464.70

### 2B. Preparation of 5-{3-[5-tert-butyl-2-(4-fluoro-phenyl)-2H-pyrazol-3-yl]-ureido}-3-chloro-4-methyl-thiophene-2-carboxylic acid

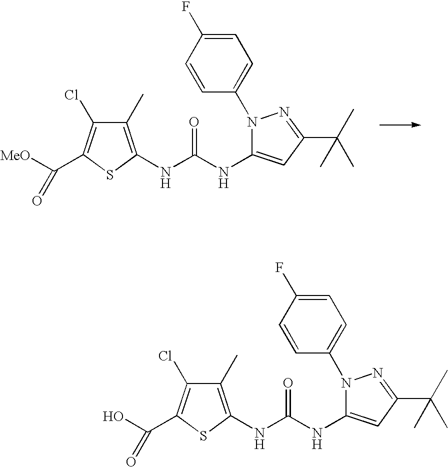

To a solution of the urea of Example 2A (0.5 g, 1 mmol) in a mixture of tetrahydrofuran:methanol:water [2:2:1] (20 ml) was added lithium hydroxide (0.36 g, 8.6 mmol) and the reaction mixture was heated at 50° C. for 36 hours. The reaction mixture was evaporated to dryness under reduced pressure and the residue was partitioned between ethyl acetate and water. The aqueous layer was acidified using 2N HCl, extracted with ethyl acetate, dried (MgSO₄), filtered and evaporated under reduced pressure to give the crude title product as a dark orange solid (0.3 g) which was used immediately in the next step without purification; LC MS M+H 450.70

### 2C. Preparation of 1-[5-tert-Butyl-2-(4-fluoro-phenyl)-2H-pyrazol-3-yl]-3-[4-chloro-3-methyl-5-(morpholine-4-carbonyl)-thiophen-2-yl]-urea

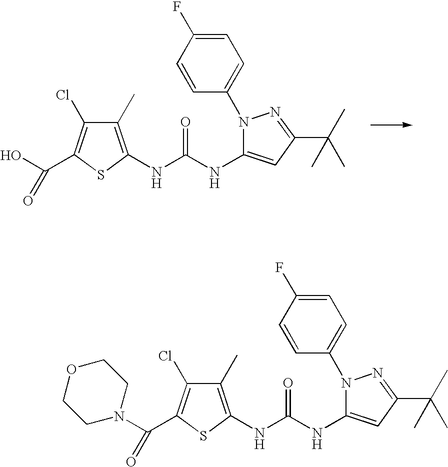

To a solution of the product of Example 2B, 5-{3-[5-tert-butyl-2-(4-fluoro-phenyl)-2H-pyrazol-3-yl]-ureido}-3-chloro-4-methyl-thiophene-2-carboxylic acid (50 mg, 0.11 mmol), in dimethyl sulphoxide (4 ml) was added EDAC (25 mg, 0.13 mmol), HOAt (18 mg, 0.13 mmol) and morpholine (10 mgs, 0.11 mmol). The reaction mixture stirred at room temperature overnight, then evaporated to dryness under reduced pressure. Purification using preparative HPLC afforded the title compound (6 mg) as a white solid; LC MS M+H 519.79

### Example 3

### 3A. Preparation of 5-(3-Fluoro-5-morpholin-4-yl-benzoylamino)-3-methyl-thiophene-2-carboxylic acid ethyl ester

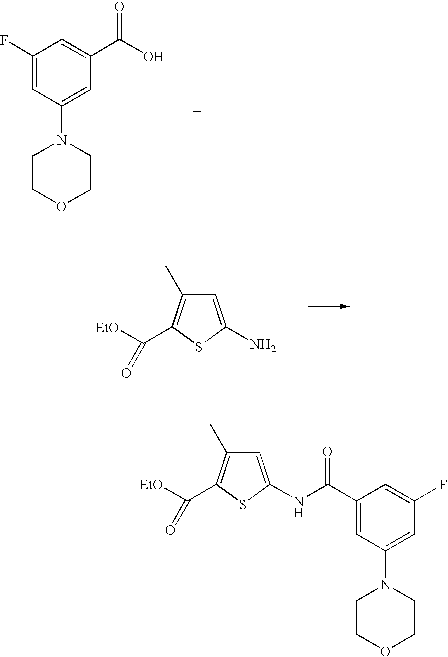

To a solution of 3-morpholino-5-fluorobenzoic acid (see Example 1A) (0.2 g, 0.88 mmol) in dichloromethane (10 ml) was added oxalyl chloride (0.11 ml, 1.08 mmol) followed by dimethylformamide (1 drop). The resulting solution was then stirred at room temperature, under an atmosphere of nitrogen, for a period of 4 hours. The solvent was then removed under reduced pressure and the residue was azeotroped to dryness by co-evaporation with toluene (2×50 ml). The residue was dissolved in dichloromethane (10 ml) and treated with 5-amino-3-methyl-2-thiophenecarboxylic acid ethyl ester (0.166 g, 0.9 mmol), triethylamine (0.1 ml, 1.8 mmol) and stirred at room temperature overnight. The reaction was diluted with dichloromethane, washed with 5% citric acid, saturated bicarbonate and saturated brine solution. The organic layers were dried (MgSO₄), filtered and evaporated under reduced pressure. The residue was recrystallised from a mixture of ethyl acetate and petroleum ether to afford the title compound (0.2 g); LC MS M+H 392.71

### 3B. Preparation of 5-(3-Fluoro-5-morpholin-4-yl-benzoylamino)-3-methyl-thiophene-2-carboxylic acid

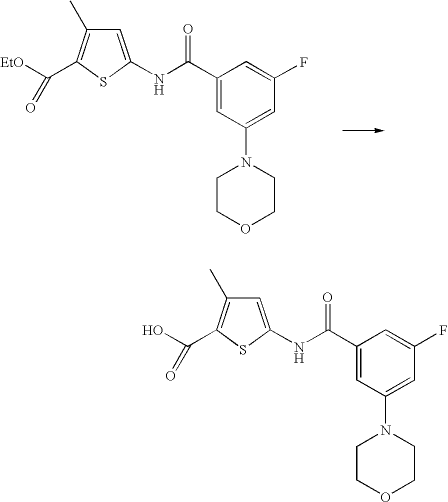

A solution of the ester of Example 3A (0.197 g, 0.5 mmol) in a mixture of tetrahydrofuran: methanol: water [2:2:1] (5 ml) was treated with lithium hydroxide (80 mg, 1.9 mmol ) and heated at 50° C. for 4 hours. The reaction mixture was evaporated to dryness and suspended in a mixture of tetrahydrofuran:water [1:1] (5 ml) and heated at 60° C. for 48 hours. The reaction mixture was evaporated and the residue was partitioned between ethyl acetate and water. The aqueous layer was acidified with 2N HCl, extracted with ethyl acetate, and the organic layers were dried (MgSO₄), filtered and evaporated to give an orange oil. Purification using prep LC afforded the title compound (60mg); LC MS M+H 364.7

### 3C. Preparation of 3-Fluoro-N-[4-methyl-5-(morpholin-4-carbonyl)-thiophen-2-yl]-5-mopholin-4-yl-benzamide

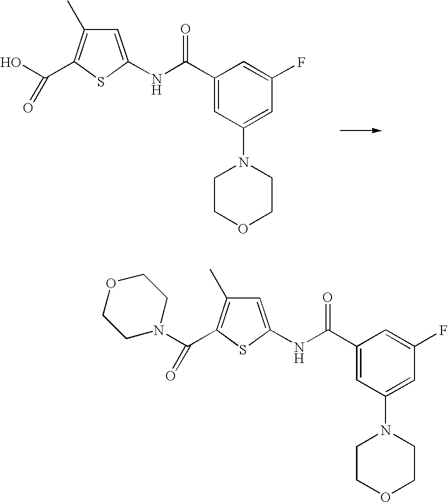

A solution of the product of Example 3B, 5-(3-Fluoro-5-morpholin-4-yl-benzoylamino)-3-methyl-thiophene-2-carboxylic acid, (27 mg, 0.07 mmol) in dimethyl sulphoxide (1 ml) was treated with EDAC (15.9 mg, 0.082 mmol), HOAt (11.2 mg,0.082 mmol) and morpholine (6.5 mg,0.07 mmol). The reaction mixture was stirred at room temperature overnight then evaporated to dryness under reduced pressure. Purification using preparative LC afforded the title compound as a white solid (4 mg); LS MS M+H 433.7

### Examples 4-18

By following the synthetic procedures set out in Examples 1 to 3, and using the appropriately substituted starting materials, the following compounds were prepared.

### Example 4

### 5-{3-[5-tert-butyl-2-(4-fluoro-phenyl)-2H-pyrazol-3-yl]-ureido}-thiophene-2-carboxylic acid ethyl ester

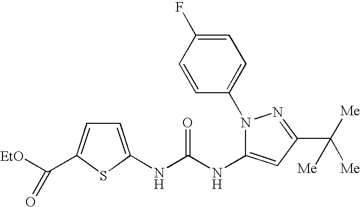

The title compound was prepared from 5-aminothiophene-2-carboxylic acid ethyl ester and 5-tert-butyl-2-(4-fluorophenyl)-2H-pyrazol-3-ylamine following the procedure described in Example 2A. LC MS M+H 431.5

### Example 5

### 1-[5-tert-Butyl-2-(4-fluorophenyl)-2H-pyrazol-3-yl]-3-[5-(morpholine-4-carbonyl)-thiophen-2-yl]-urea

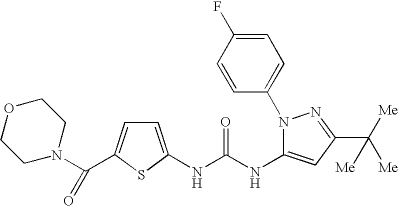

The title compound was prepared from 5-{3-[5-tert-butyl-2-(4-fluoro-phenyl)-2H-pyrazol-3-yl]-ureido}-thiophene-2-carboxylic acid and morpholine following the procedures set out in examples 2B and 2C. LC MS M+H 472.6

### Example 6

### 5-{3-[5-tert-butyl-2-(4-fluoro-phenyl)-2H-pyrazol-3-yl]-ureido}-3-methyl-4-cyano-thiophene-2-carboxylic acid methyl ester

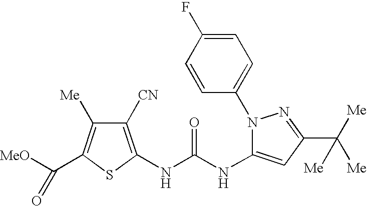

This compound was prepared from 3-methyl-4-cyano-5-aminothiophene-2-carboxylic acid ethyl ester and 5-tert-butyl-2-(4-fluorophenyl)-2H-pyrazol-3-ylamine following the procedure described in Example 2A. LC MS M+H 456.6

### Example 7

### 3-Cyano-5-(4-fluorobenzoylamino)-4-methyl-thiophene-2-carboxylic acid methyl ester

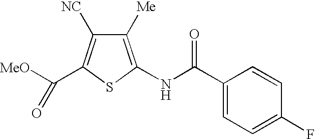

The title compound was prepared from 3-cyano-4-methyl-5-aminothiophene-2-carboxylic acid methyl ester and 4-fluorobenzoic acid using the method of Example 1C. LC MSM+H319.3

### Example 8

### N-[4-Chloro-3-methyl-5-(morpholine-4-carbonyl)-thiophen-2-yl]-4-fluorobenzamide

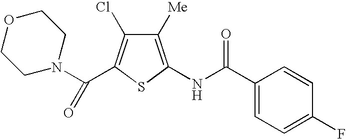

The title compound was prepared from 3-chloro-5-(4-fluorobenzoylamino)-4-methyl-thiophene-2-carboxylic acid and morpholine using the procedure of Example 1E. LC MS M+H 383.9

### Example 9

### N-[4-Chloro-3-methyl-5-(4-fluorophenylaminocarbonyl)-thiophen-2-yl]-4-fluorobenzamide

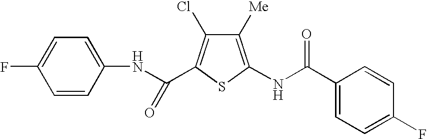

The title compound was prepared from 3-chloro-5-(4-fluorobenzoylamino)-4-methyl-thiophene-2-carboxylic acid and 4-fluoroaniline using the procedure of Example 1E. LC MS M+H 407.9

### Example 10

### 3-Chloro-5-(4-fluorobenzoylamino)4-methyl-thiophene-2-carboxylic acid methyl ester

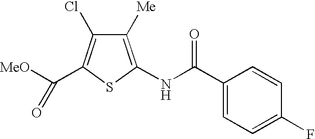

This compound was prepared from 3-chloro-5-amino-4-methyl-thiophene-2-carboxylic acid methyl ester and 4-fluorobenzoic acid using the procedure of Example 1C. LC MS M+H 328.8

### Example 11

### 1-[5-tert-Butyl-2-(4-fluoro-phenyl)-2H-pyrazol-3-yl]-3-[4-chloro-3-methyl-5-(1-methylpiperazine-4-carbonyl)-thiophen-2-yl]-urea

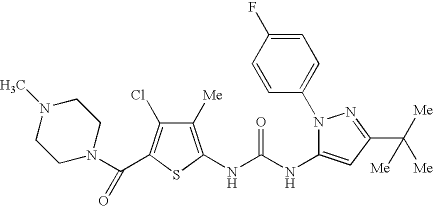

The title compound was prepared by reacting 5-{3-[5-tert-butyl-2-(4-fluoro-phenyl)-2H-pyrazol-3-yl]-ureido}-3-chloro-4-methyl-thiophene-2-carboxylic acid with 1-methyl piperazine using the procedure of Example 2C. LC-MS M+H 534.1

### Example 12

### 1-[5-tert-Butyl-2-(4-fluoro-phenyl)-2H-pyrazol-3-yl]-3-[4-chloro-3-methyl-5-(4-pyridylmethylaminocarbonyl)-thiophen-2-yl]-urea

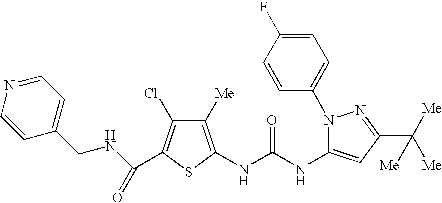

The title compound was prepared by reacting 5-{3-[5-tert-butyl-2-(4-fluoro-phenyl)-2H-pyrazol-3-yl]-ureido}-3-chloro-4-methyl-thiophene-2-carboxylic acid with 4-pyridylmethylamine using the procedure of Example 2C. LC-MS M+H 542.1

### Example 13

### N-[4-Chloro-3-methyl-5-(4-pyridylmethylaminocarbonyl)-thiophen-2-yl]-3-fluoro-5-morpholin-4-yl-benzamide

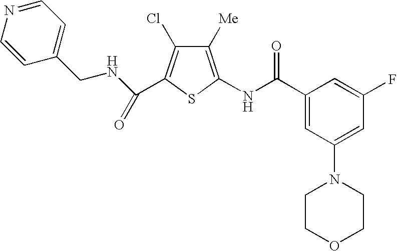

The title compound was prepared by reacting 3-chloro-5-(3-fluoro-5-morpholin-4-yl-benzoylamino)-4-methyl-thiophene-2-carboxylic acid with 4-pyridylmethyl-amine using the procedure of Example 1E. LC MS M+H 490

### Example 14

### N-[4-Chloro-3-methyl-5-(2,3,5-trimethyl-2H-pyrazol-4-ylaminocarbonyl)-thiophen-2-yl]-3-fluoro-5-morpholin-4-yl-benzamide

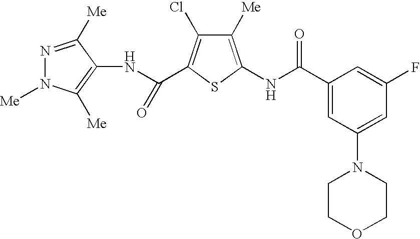

The title compound was prepared by reacting 3-chloro-5-(3-fluoro-5-morpholin-4-yl-benzoylamino)-4-methyl-thiophene-2-carboxylic acid with 4-amino-2,3,5-trimethyl-2H-pyrazole using the procedure of Example 1E. LC MS M+H 507

### Example 15

### N-[4-Chloro-3-methyl-5-(4-fluorophenylaminocarbonyl)-thiophen-2-vl]-3-fluoro-5-morpholin-4-yl-benzamide

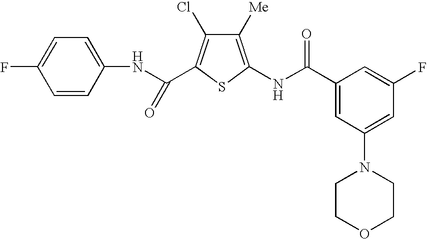

The title compound was prepared by reacting 3-chloro-5-(3-fluoro-5-morpholin-4-yl-benzoylamino)-4-methyl-thiophene-2-carboxylic acid with 4-fluoroaniline using the procedure of Example 1E. LC MS M+H 493

### Example 16

### N-[4-Chloro-3-methyl-5-(3-amino-1H-1,2,4-triazol-5-ylaminocarbonyl)-thiophen-2-yl]-3-fluoro-5-morpholin-4-yl-benzamide

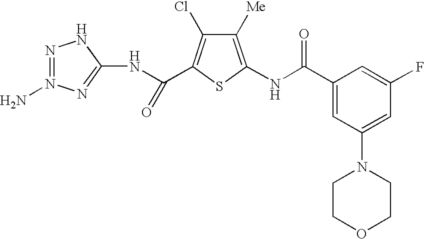

The title compound was prepared by reacting 3-chloro-5-(3-fluoro-5-morpholin-4-yl-benzoylamino)-4-methyl-thiophene-2-carboxylic acid with 3,5 diamino-1H-1,2,4-triazole using the procedure of Example 1E. LC MS M+H 481

### Example 17

### N-[4-Chloro-3-methyl-5-(1-methylpiperazin-4-ylaminocarbonyl)-thiophen-2-yl]-3-fluoro-5-morpholin-4-yl-benzamide

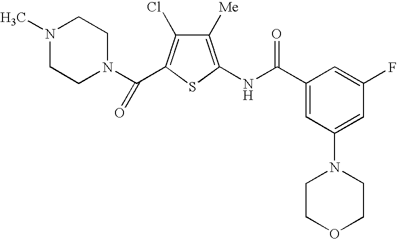

The title compound was prepared by reacting 3-chloro-5-(3-fluoro-5-morpholin-4-yl-benzoylamino)-4-methyl-thiophene-2-carboxylic acid with N-methylpiperazine using the procedure of Example 1E. LC MS M+H 482

### Example 18

### N-[4-Chloro-3-methyl-5-(2-amino-primidin-5-ylaminocarbonyl)-thiophen-2-yl]-3-fluoro-5-morpholin-4-yl-benzamide

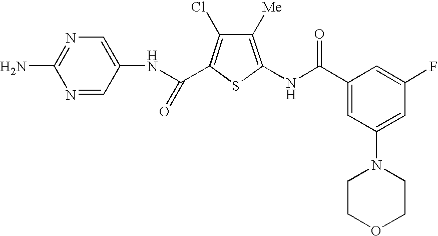

The title compound was prepared by reacting 3-chloro-5-(3-fluoro-5-morpholin-4-yl-benzoylamino)-4-methyl-thiophene-2-carboxylic acid with 2,5-diaminopyrimidine using the procedure of Example 1E. LC MS M+H 492

### Example 19

### 19A. Preparation of (3-chloro-4-methyl-thiophen-2-yl)-morpholin-4-yl-methanone

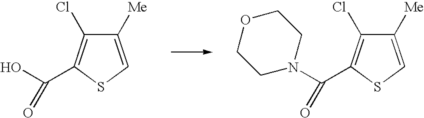

To a solution of 3-chloro-4-methyl-thiophen-2-carboxylic acid (2 g, 1.13 mmol) in dichloromethane (70 ml) was added EDAC (2.56 g, 1.3 mmol), HOBt (2 g, 1.3 mmol) followed by morpholine (1 ml, 1.2 mmol). The reaction mixture was stirred at room temperature overnight and then diluted with dichloromethane (50 ml). The diluted reaction mixture was washed with 5% citric acid solution (30 ml) and brine (30 ml), dried (MgSO₄), filtered and the solvent was removed under reduced pressure to afford the crude product (1.5 g) which was used immediately in the next step without purification); LC MS M+H 246.

### 19B. Preparation of (5-amino-3-chloro-4-methyl-thiophen-2-yl)-morpholin-4-yl-methanone

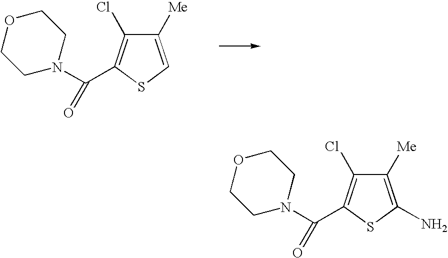

To a solution of (3-chloro-4-methyl-thiophen-2-yl)-morpholin-4-yl-methanone (0.94 g, 3.8 mmol) in acetonitrile (100 ml) was added nitronium tetrafluoroborate at 0° C. The reaction mixture was stirred for 18 hours, then diluted with water (100 ml) and extracted with dichloromethane (200 ml). The organic solution was washed with saturated sodium bicarbonate solution (50 ml) and brine (50 ml), dried (MgSO₄), filtered and the solvent removed under reduced pressure to afford the crude product (1.1 g) as an orange oil, which was used immediately in the next step without purification.

To a solution of the crude (3-chloro-4-methyl-5-nitro-thiophen-2-yl)-morpholin-4-yl-methanone (1.1 g, 0.37 mmol) in dioxane (25 ml) and water (5 ml) was added iron powder (1.9 g) followed by iron sulphate heptahydrate (2.3 g). The reaction mixture was then heated to reflux for 4 hours before cooling to room temperature. The solvent was then removed under reduced pressure and the residue was partitioned between ethyl acetate (15 ml) and 1N HCl (10 ml). The organic layer was separated and the aqueous layer was basified with saturated sodium bicarbonate solution. The solution was extracted with ethyl acetate (2×25 ml), the organic layers were combined, dried (MgSO₄), filtered and the solvent was removed under reduced pressure. The residue was subjected to purification by flash column chromatography on silica gel, eluting with ethyl acetate/petroleum ether mixtures to afford the title compound as a brown oil (1.77 g, 18% over two steps); LC MS M+H 261.

### 19C. Preparation of 1-[4-chloro-3-methyl-5-(morpholine-4-carbonyl)-thiophen-2-yl]-3-(5-cyclopropylmethyl-[1,3,4]thiadiazol-2-yl)-urea

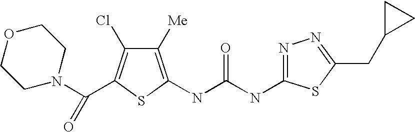

To a stirred solution of (5-amino-3-chloro-4-methyl-thiophen-2-yl)-morpholin-4-yl-methanone in dry dichloromethane (5 ml) was added 20% phosgene in toluene (0.4 ml) at room temperature and the reaction mixture was stirred for 48 hours. 5-Cyclopropylmethyl-[1,3,4]thiadiazol-2-ylamine (30 mg, 0.19 mmol) was added dropwise in dry dichloromethane (2 ml) and the reaction mixture was stirred at room temperature for 18 hours. The reaction mixture was diluted with methanol (2 ml) and the solvent was removed under reduced pressure. The residue was subjected to purification by flash column chromatography on silica gel, eluting with 2% methanol/dichloromethane to afford the title compound as a solid (40 mg); LC MS M+H 443.

### Example 20

### 1-[2-(Tetrahydrofuran-2-yl)-thiadiazol-5-yl]-3-[4-chloro-3-methyl-5-(morpholine-4-carbonyl)-thiophen-2-yl]-urea

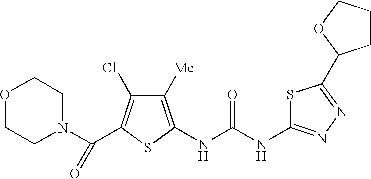

The title compound was prepared by reacting (5-amino-3-chloro-4-methyl-thiophen-2-yl)-morpholin-4-yl-methanone with phosgene and subsequently with 5-(tetrahydrofuran-2-yl)-[1,3,4]thiadiazol-2-ylamine following the procedures set out in Example 19C. LC MS M+H 459

### Example 21

### 1-[4-chloro-3-methyl-5-morpholine-4-carbonyl)-thiophen-2-yl]-3-[5-(4-fluoro-benzyl)-[1,3,4]thiadiazol-2-yl]-urea

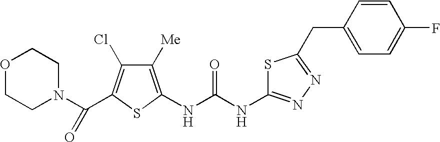

The title compound was prepared by reacting (5-amino-3-chloro-4-methyl-thiophen-2-yl)-morpholin-4-yl-methanone with phosgene and subsequently with 5-(4-fluorobenzyl)-[1,3,4]thiadiazol-2-ylamine following the procedures set out in Example 19C. LC MS M+H 497

### Example 22

### 1-[4-chloro-3-methyl-5-(morpholine-4-carbonyl)-thiophen-2-yl]-3-[5-cyclohexyl-[1,3,4]thiadiazol-2-yl]-urea

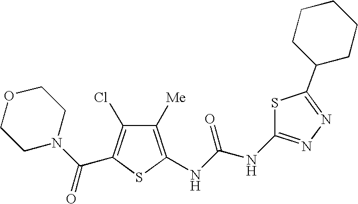

The title compound was prepared by reacting (5-amino-3-chloro-4-methyl-thiophen-2-yl)-morpholin-4-yl-methanone with phosgene and subsequently with 5-(cyclohexyl)-[1,3,4]thiadiazol-2-ylamine following the procedures set out in Example 19C. LC MS M+H 471.1

### Example 23

### 1-[4-chloro-3-methyl-5-(morholine-4-carbonyl)-thiophen-2-yl]-3-(5-morpholin-4-yl-[1,3,4]thiadiazol-2-yl)-urea

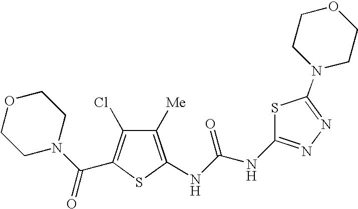

The title compound was prepared by reacting (5-amino-3-chloro-4-methyl-thiophen-2-yl)-morpholin-4-yl-methanone with phosgene and subsequently with 5-(morpholin-4-yl)-[1,3,4]thiadiazol-2-ylamine following the procedures set out in Example 19C. LC MS M+H 474

### Example 24

### 1-[4-chloro-3-methyl-5-(morpholine-4-carbonyl)-thiophen-2-yl]-3-[5-(4-methyl-piperazin-1-yl)-[1,3,4]thiadiazol-2-yl]-urea

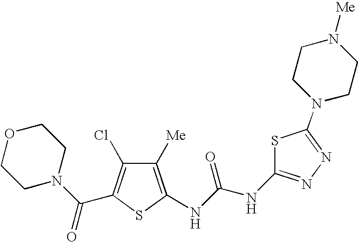

The title compound was prepared by reacting (5-amino-3-chloro-4-methyl-thiophen-2-yl)-morpholin-4-yl-methanone with phosgene and subsequently with 5-(4-methyl-piperazin-1-yl)-[1,3,4]thiadiazol-2-ylamine following the procedures set out in Example 19C. LC MS M+H 487.1

### Example 25

### 1-[5-tert-Butyl-2-(2,4-difluoro-phenyl)-2H-pyrazol-3-yl]-3-[4-chloro-3-methyl-5-(morpholine-4-carbonyl)-thiophen-2-yl]-urea

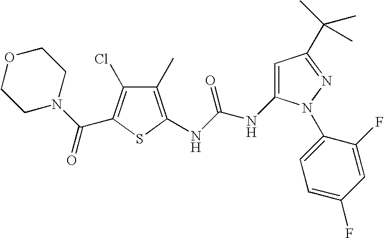

The title compound was prepared by reacting (5-amino-3-chloro-4-methyl-thiophen-2-yl)-morpholin-4-yl-methanone with phosgene and subsequently with 5-tert-butyl-2-(2,4-difluoro-phenyl)-2H-pyrazol-3-ylamine following the procedures set out in Example 19C. LC MS M+H 539.1

### Biological Activity

### Example 26

p38 MAP Kinase Inhibitory Activity

Measurement of p38 MAP Kinase Inhibitory Activity (IC₅₀)

Compounds of the invention were tested for p38 MAP kinase inhibitory activity using the protocol set out below.

In the assay, an inactive a isoform of p38 mitogen-activated protein kinase was used. The structure of this kinase at 2.1-A resolution is described in the article by Wang Z, Harkins P C, Ulevitch R J, Han J, Cobb M H and Goldsmith E J. in Proc. Natl. Acad. Sci. USA 1997 Mar. 18;94(6):2327. The a isoform of p38 MAP kinase was activated using the MKK6 kinase obtained from Upstate Biotechnology. The selective activation of p38 mitogen-activated protein (MAP) kinase isoforms by the MAP kinase kinase MKK6 is described in the article by Enslen, H; Raingeaud, J and Davis, R J in The Journal of Biological Chemistry, Volume 273, Issue 3, Jan. 16, 1998, Pages 1741-1748.

The protocol was as follows:

1 ml of fresh assay buffer (25 mM HEPES pH 7.4, 25 mM β-glycerophosphate, 5 mM EDTA, 15 mM MgCl₂, 100 μM ATP, 1 mM sodium orthovanadate, 1 mM DTT), 35 μg of inactive purified a p38 and 0.12 μg of active MKK6 (1688 U/mg—Upstate Biotechnology) are mixed and incubated at room temperature overnight to activate the p38. The activated p38 is then diluted sixfold with assay buffer without ATP, and 10 μl mixed with 5 μl of various dilutions of the test compound in DMSO (up to 1.7%) in a 96 well plate and incubated at room temperature for 1.5 hours. Next, 10 μl of MBP mix (150 μl 10× strength assay buffer (250 mM HEPES pH 7.4, 250 mM β-glycerophosphate, 50 mM EDTA, 150 mM MgCl₂), 1.5 μl of 10 mM DTT & 10 mM sodium orthovanadate, 17.5 μl of 10 mM ATP, 713 μl H₂O, 35 μCi γ³³P-ATP, 100 μl of myelin basic protein (MBP) (5 mg/ml)) is added to each well. MBP is a protein of bovine origin having a molecular weight of 18.4 kDa and is obtained from Upstate Biotechnology. The reaction is allowed to proceed for 50 minutes before being stopped with an excess of ortho-phosphoric acid (5 μl at 12.5%).

γ³³P-ATP which remains unincorporated into the myelin basic protein is separated from phosphorylated MBP on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 25 μl of Microscint 20™ scintillant is added, and then counted on a Packard Topcount for 30 seconds. The % inhibition of the p38 activity is calculated and plotted in order to determine the concentration of test compound required to inhibit 50% of the p38 activity (IC₅₀).

The compounds of Examples 1C, 1E, 2A, 2C, 3A, 3C and 4 to 18 were tested using the assay and all were found to inhibit p38 activity. The compounds of Examples 1C, 1E, 2A, 2C, 3C, 4 to 9, 11, 12, 15, 22, 23 and 25 all had IC₅₀ values of less than 15 μM.

### Example 26

Inhibition of LPS-Induced INF-α Production in THP-1 Cells, In Vitro Assay

The ability of the compounds of this invention to inhibit the INF-α release may be determined using a minor modification of the methods described in Rawlins P., et al., “Ihibition of endotoxin-induced TNF-α production in macrophages by 5Z-7-oxo-zeaenol and other fungal resorcyclic acid lactones,” International J. of Immunopharmacology, 21, 799, (1999).

THP-1 cells, human monocytic leukaemic cell line, ECACC) are maintained in culture medium [RPMI 1640 (Invitrogen) and 2 mM L-Glutamine supplemented with 10% foetal bovine serum (Invitrogen)] at approximately 37° C. in humidified 5% CO₂ in stationary culture.

THP-1 cells are suspended in culture medium containing 50 ng/ml PMA (SIGMA), seeded into a 96-well tissue culture plate (IWAKI) at 1×10⁵ cells/well (100 μl/well) and incubated as described above for approximately 48 h. The medium is then aspirated, the wells washed twice in Phosphate Buffered Saline and 1 g/ml LPS (SIGMA) in culture medium is added (200 μl/well).

Test compounds are reconstituted in DMSO (SIGMA) and then diluted with the culture medium such that the final DMSO concentration is 0.1%. Twenty microlitre aliquots of test solution or medium only with DMSO (solvent control) are added to triplicate wells immediately following LPS addition, and incubated for 6 h as described above. Culture supernatants are collected and the amount of human TNF-α present is determined by ELISA (R&D Systems) performed according to the manufacturer's instructions.

The IC₅₀ is defined as the concentration of the test compound corresponding to half maximal inhibition of the control activity by non-linear regression analysis of their inhibition curves.

### Pharmaceutical Formalations

### Example 27

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

(iii) Aerosol Formulation

An aerosol formulation for administration by inhalation is prepared by weighing micronised compound of the formula (I) (60 mg) directly into an aluminium can and then adding 1,1,1,2-tetrafluorethane (to 13.2 g) from a vacuum flask. A metering valve is crimped into place and the sealed can is sonicated for five minutes. The resulting formulation delivers the compound of formula (I) as an aerosol in an amount of 250 mg of per actuation.

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-80. (canceled)

81. A method for the prophylaxis or treatment of a disease state or condition mediated by a p38 MAP kinase, which method comprises administering to a subject in need thereof a therapeutically effective amount of a compound of the formula (I): 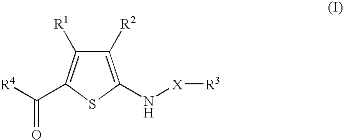
or a salt, solvate or N-oxide thereof, wherein:
R¹ and R² are the same or different and each is selected from hydrogen, C₁₋₄ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members, the aryl and heteroaryl groups each being unsubstituted or substituted by one or more substituent groups R⁷;
R⁷ is selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X2)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
X¹ is O, S or NR^{c} and X² is ═O, ═S or ═NR^{c};
R^{c} is hydrogen or C₁₋₄ hydrocarbyl;
R⁴ is a group YR⁵ or a group R⁶;
Y is is NH, O or S;
R⁵ is selected from (a) carbocyclic and heterocyclic groups having from 3 to 12 ring members; and (b) C₁₋₈ hydrocarbyl groups optionally substituted by one or more substituents selected from hydroxy, oxo, halogen, cyano, amino, mono- or di-C₁₋₄ hydrocarbylamino, and carbocyclic and heterocyclic groups having from 3 to 12 ring members, wherein one or more carbon atoms of the C₁₋₈ hydrocarbyl group may optionally be replaced by O, S, SO, SO₂, NR^{c}, X¹C(X²), C(X²)X¹ or X¹C(X²)X¹, provided that when Y is O, a carbon atom adjacent to the group Y is not replaced by O; and
R⁶ is a heterocyclic group having from 4 to 12 ring members and containing at least one ring nitrogen atom through which R⁶ is linked to the adjacent carbonyl group;
wherein the carbocyclic and heterocyclic groups of substituents R⁵ and R⁶ are each unsubstituted or substituted by one or more substituent groups R⁷ as hereinbefore defined.

82. A method according to claim 81 wherein the disease state or condition is selected from inflammatory and arthritic diseases and conditions.

83. A compound of the formula (Ia): 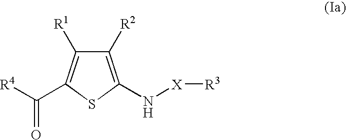
or a salt, solvate or N-oxide thereof, wherein:
R¹ and R² are the same or different and each is selected from hydrogen, C₁₋₄ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members, the aryl and heteroaryl groups each being unsubstituted or substituted by one or more substituent groups R⁷;
R⁷ is selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
X¹ is O, S or NR^{c} and X² is ═O, ═S or ═NR^{c};
R^{c} is hydrogen or C₁₋₄ hydrocarbyl;
R⁴ is a group YR⁵ or a group R⁶;
Y is is NH, O or S;
R⁵ is selected from (a) carbocyclic and heterocyclic groups having from 3 to 12 ring members; and (b) C₁₋₈ hydrocarbyl groups optionally substituted by one or more substituents selected from hydroxy, oxo, halogen, cyano, amino, mono- or di-C₁₋₄ hydrocarbylamino, and carbocyclic and heterocyclic groups having from 3 to 12 ring members, wherein one or more carbon atoms of the C₁₋₈ hydrocarbyl group may optionally be replaced by O, S, SO, SO₂, NR^{c}, X¹C(X²), C(X²)X¹ or X¹C(X²)X¹, provided that when Y is O, a carbon atom adjacent to the group Y is not replaced by O; and
R⁶ is a heterocyclic group having from 4 to 12 ring members and containing at least one ring nitrogen atom through which R⁶ is linked to the adjacent carbonyl group, provided that R⁶ is other than a bicyclic group comprising a benzene ring fused to a 7-membered heterocyclic ring;
wherein the carbocyclic and heterocyclic groups of substituents R⁵ and R⁶ are each unsubstituted or substituted by one or more substituent groups R⁷ as hereinbefore defined;
provided that:
(a) when X is C═O and R³ is a heteroaryl group substituted by the group R^{a}—R^{b} where R^{a} is NR^{c}C═O, then R^{b} is other than an optionally further substituted phenyl, pyridyl or pyrimidinyl group having a carbocyclic or heterocylic group bonded to the ortho position thereof either directly or through an intervening linker atom or group of 1 or 2 atoms in length;
(b) when X is C═O, R³ is other than:
(i) an optionally further substituted phenyl, pyridyl or pyrimidinyl group having a carbocyclic or heterocylic group bonded to the ortho position thereof either directly or through an intervening linker atom or group of 1 or 2 atoms in length;
(ii) a phenyl group having an oxy-substituent bonded to the ortho position thereof;
(iii) an optionally N-substituted pyrrolidine ring substituted on a carbon atom thereof by a group selected from thiol, substituted thiol, thiocarbonate and groups containing a β-lactam ring;
(c) when X is C═O and R³ is an unsubstituted phenyl group, or a phenyl group substituted by one or more substituents, none of which are cyclic, then R⁴ is other than alkoxy;
(d) when X is C(═O)NH and R³ is a thiophene group bearing a 5-alkoxycarbonyl group, then R⁴ is other than alkoxy;
(e) when Y is NH or O and R⁵ is a C₂₋₄ alkylene group bearing a terminal amino, monoalkylamino or dialkylamino substituent, wherein the alkyl moieties of the mono- and dialkylamino substituents are themselves unsubstituted or further substituted; then X—R³is other than an unsubstituted or substituted benzoyl group;
(f) when Y is NH and R⁵ is a C₁₋₃ alkylene group bearing a terminal carboxy or alkoxycarbonyl substituent; then X—R³ is other than a 4-carbamimidoyl-benzoyl group;
(g) when X is C═O, Y is NH and R⁵ is a 3-dimethylaminoprop-1-yl group; then R³ is other than a 5-nitro-2-thiophenyl group; and
(h) when X is C═O, R⁴ is ethoxy, R¹ is methyl and R² is hydrogen or cyano; then R³ is other than an unsubstituted phenyl group.

84. A compound according to claim 83 wherein X is selected from C═O and C(═O)NH.

85. A compound according to claim 84 wherein R³ is a monocyclic aryl or heteroaryl group, which monocyclic aryl or heteroaryl group is unsubstituted or substituted by one or more substituent groups R⁷.

86. A compound according to claim 85 wherein the monocyclic aryl and heteroaryl group is selected from phenyl, pyrazolyl, and thiadiazolyl groups, wherein the phenyl, pyrazolyl, and thiadiazolyl groups are each unsubstituted or substituted by one or more substituent groups R⁷.

87. A compound according to claim 86 wherein the monocyclic aryl group or heteroaryl group R³ contains one or more substituent groups R⁷ selected from halogen, carbocyclic and heterocyclic groups having from 4 to 7 ring members and optionally substituted C₁₋₈ hydrocarbyl groups.

88. A compound according to claim 87 wherein one of said one or more substituent groups R⁷ is a carbocyclic or heterocyclic group which is linked to the aryl or heteroaryl ring via a carbon nitrogen bond.

89. A compound according to claim 88 in which R⁴ is a group R⁶ wherein R⁶ is a monocyclic group having from 4 to 7 ring members.

90. A compound according to claim 89 wherein the monocyclic group R⁶ is a group: 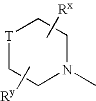
where T is N-methyl or O; R^{x} and R^{y} are the same or different and are selected from hydrogen and methyl; or one of R^{x} and R^{y} is selected from hydroxymethyl and ethyl and the other is hydrogen.

91. A compound according to clam 90 wherein T is O and R^{x} and R^{y} are both hydrogen.

92. A compound according to claim 91 containing a combination of groups R¹ and R² selected from: (a) R¹=chlorine and R²=methyl; (b) R¹=chlorine and R²=hydrogen; (c) R¹=hydrogen and R²=hydrogen; (d) R¹=methyl and R²=hydrogen; (e) R¹=cyano and R²=methyl; and (f) R¹=methyl and R²=cyano.

93. A compound according to claim 92 wherein the combination of groups R¹ and R² is combination (a).

94. A compound according to claim 92 wherein X is C═O.

95. A compound according to claim 94 wherein R³ is a phenyl group bearing one or two meta substituents.

96. A compound according to claim 95 wherein one meta position on the phenyl ring is unsubstituted or is substituted by a group selected from fluorine, chorine, methoxy, trifluoromethoxy, trifluoromethyl, ethyl, methyl and isopropyl; and the other meta position is substituted by a group selected from fluorine, chorine, methoxy, trifluoromethoxy, trifluoromethyl, ethyl, methyl, isopropyl, isobutyl, t-butyl, phenyl, substituted phenyl, and five and six membered monocyclic heterocyclic groups.

97. A compound according to claim 96 wherein both meta positions on the phenyl ring are substituted, one substituent being a halogen and the other substituent being a morpholine group.

98. A compound according to claim 92 wherein X is C(═O)NH.

99. A compound according to claim 98 wherein R³ is a pyrazole group substituted by two substituent groups R⁷.

100. A compound according to claim 99 wherein the two substituent groups R⁷ are located on non-adjacent ring members.

101. A compound according to claim 100 wherein the pyrazole group is substituted by an optionally substituted phenyl group and a C₁₋₄ hydrocarbyl group.

102. A compound according to claim 101 wherein the optionally substituted phenyl group is 4-fluorophenyl.

103. A compound according to claim 101 wherein the C₁₋₄ hydrocarbyl group is tert-butyl.

104. A compound according to claim 83 selected from:
3-chloro-5-(3-fluoro-5-morpholin-4-yl-benzoylamino)-4-methyl-thiophene-2-carboxylic acid methyl ester;
N-[4-chloro-3-methyl-5-(morpholine-4-carbonyl)-thiophen-2-yl]-3-fluoro-5-morpholin-4-yl-benzamide;
5-{3-[5-tert-butyl-2-(4-fluoro-phenyl)-2H-pyrazol-3-yl]-ureido}-3-chloro-4-methyl-thiophene-2-carboxylic acid methyl ester;
1-[5-tert-butyl-2-(4-fluoro-phenyl)-2H-pyrazol-3-yl]-3-[4-chloro-3-methyl-5-(morpholine-4-carbonyl)-thiophen-2-yl]-urea;
5-(3-fluoro-5-morpholin-4-yl-benzoylamino)-3-methyl-thiophene-2-carboxylic acid ethyl ester;
3-fluoro-N-[4-methyl-5-(morpholin-4-carbonyl)-thiophen-2-yl]-5-morpholin-4-yl-benzamide;
5-{3-[5-tert-butyl-2-(4-fluoro-phenyl)-2H-pyrazol-3-yl]-ureido}-thiophene-2-carboxylic acid ethyl ester;
1-[5-tert-butyl-2-(4-fluorophenyl)-2H-pyrazol-3-yl]-3-[5-(morpholine-4-carbonyl)-thiophen-2-yl]-urea;
5-{3-[5-tert-butyl-2-(4-fluoro-phenyl)-2H-pyrazol-3-yl]-ureido}-3-methyl-4-cyano-thiophene-2-carboxylic acid methyl ester;
3-cyano-5-(4-fluorobenzoylamino)-4-methyl-thiophene-2-carboxylic acid methyl ester;
N-[4-chloro-3-methyl-5-(morpholine-4-carbonyl)-thiophen-2-yl]-4-fluorobenzamide;
N-[4-chloro-3-methyl-5-(4-fluoro-phenylaminocarbonyl)-thiophen-2-yl]-4-fluorobenzamide;
3-chloro-5-(4-fluorobenzoylamino)-4-methyl-thiophene-2-carboxylic acid methyl ester;
1-[5-tert-butyl-2-(4-fluoro-phenyl)-2H-pyrazol-3-yl]-3-[4-chloro-3-methyl-5-(1-methylpiperazine-4-carbonyl)-thiophen-2-yl]-urea;
1-[5-tert-butyl-2-(4-fluoro-phenyl)-2H-pyrazol-3-yl]-3-[4-chloro-3-methyl-5-(4-pyridylmethylaminocarbonyl)-thiophen-2-yl]-urea;
N-[4-chloro-3-methyl-5-(4-pyridylmethylaminocarbonyl)-thiophen-2-yl]-3-fluoro-5-morpholin-4-yl-benzamide;
N-[4-chloro-3-methyl-5-(2,3,5-trimethyl-2H-pyrazol-4-ylaminocarbonyl)-thiophen-2-yl]-3-fluoro-5-morpholin-4-yl-benzamide;
N-[4-chloro-3-methyl-5-(4-fluorophenylaminocarbonyl)-thiophen-2-yl]-3-fluoro-5-morpholin-4-yl-benzamide;
N-[4-chloro-3-methyl-5-(1-methylpiperazin-4-ylaminocarbonyl)-thiophen-2-yl]-3-fluoro-5-morpholin-4-yl-benzamide;
N-[4-chloro-3-methyl-5-(2-amino-pyrimidin-5-ylaminocarbonyl)-thiophen-2-yl]-3-fluoro-5-morpholin-4-yl-benzamide;
1-[2-(tetrahydrofuran-2-yl)-thiadiazol-5-yl]-3-[4-chloro-3-methyl-5-(morpholine-4-carbonyl)-thiophen-2-yl]-urea;
1-[4-chloro-3-methyl-5-(morpholine-4-carbonyl)-thiophen-2-yl]-3-[5-cyclohexyl-[1,3,4]thiadiazol-2-yl]-urea;
1-[4-chloro-3-methyl-5-(morpholine-4-carbonyl)-thiophen-2-yl]-3-(5-morpholin-4-yl-[1,3,4]thiadiazol-2-yl)-urea;
1-[4-chloro-3-methyl-5-(morpholine-4-carbonyl)-thiophen-2-yl]-3-[5-(4-methyl-piperazin-1-yl)-[1,3,4]thiadiazol-2-yl]-urea; and
1-[5-tert-Butyl-2-(2,4-difluoro-phenyl)-2H-pyrazol-3-yl]-3-[4-chloro-3-methyl-5-(morpholine-4-carbonyl)-thiophen-2-yl]-urea;
and salts, solvates and N-oxides thereof.

105. A pharmaceutical composition comprising a compound as defined in claim 83 together with a pharmaceutically acceptable carrier.
